(19) United States
(12) Patent Application Publication
(10) Pub. No.: US 2025/0339119 A1
(43) Pub. Date: Nov. 6, 2025

(54) RADIOGRAPHIC IMAGING SYSTEM

(71) Applicant: Konica Minolta, Inc., Tokyo (JP)

(72) Inventors: Kazuya Yogi, Kawagoe-shi (JP); Satoshi Hasegawa, Tokyo (JP); Nobuyuki Miyake, Yokohama-shi (JP); Tatsuya Takagi, Tokyo (JP); Naoki Hayashi, Tokyo (JP)

(73) Assignee: Konica Minolta, Inc., Tokyo (JP)

(21) Appl. No.: 19/197,448

(22) Filed: May 2, 2025

(30) Foreign Application Priority Data
May 2, 2024 (JP) 2024-074759

(51) Int. Cl. A61B 6/00; A61B 6/46

(52) U.S. Cl. CPC A61B 6/5247; A61B 6/463; A61B 6/547; A61B 6/4417

(57) ABSTRACT

A radiographic imaging system includes: an optical camera that obtains an optical image; a display that displays the optical image obtained by the optical camera; a radiation emitter that emits radiation to a radiographic imaging device that generates a radiographic image; and a hardware processor. The hardware processor calculates angle information of the radiographic imaging device in a right-left direction and an up-down direction with respect to a direction of the radiation emitted by the radiation emitter. The hardware processor superposes predetermined information on the optical image and displays the predetermined information and the optical image on the display. The predetermined information is based on the calculated angle information of the radiographic imaging device in the right-left direction and the up-down direction.

### CROSS-REFERENCE TO RELATED APPLICATIONS

The entire disclosure of Japanese Patent Application No. 2024-074759 filed on May 2, 2024, is incorporated herein by reference in its entirety.

### BACKGROUND OF THE INVENTION

### Technical Field

The present invention relates to a radiographic imaging system.

### Description of Related Art

Radiographic imaging of a subject on a bed in a hospital ward may be performed using a movable radiographic imaging system called a medical cart, for example. In performing imaging on the bed, the imaging surface of a portable (panel-like) radiographic imaging device to be disposed between the back of the subject and the bed may not be parallel or orthogonal to a horizontal plane (i.e., the imaging surface is inclined). In such a case, it is necessary to adjust the orientation of the tube to the radiographic imaging device so that the irradiation axis of the radiation is orthogonal to the imaging surface of the radiographic imaging device, in order to prevent density variations in a radiographic image owing to the cutoff of the grid attached to the radiation incident surface and to prevent changes in the positional relationship of the internal structures of the subject imaged on the radiographic imaging device from affecting diagnosis.

For example, Japanese Unexamined Patent Publication No. 2000-23955 discloses displaying the postures of the tube and the radiographic imaging device on the liquid crystal display of the tube to support adjusting the orientation of the tube (radiation source) with respect to the radiographic imaging device (cassette).

The technology disclosed in JP2000-23955A displays, as information indicating the postures, only (i) the horizontal and vertical rotation angles of the radiographic imaging device and (ii) the rotation angles with respect to the line segment connecting the tube and the radiographic imaging device as numerical values. It is difficult to intuitively grasp the postures. Therefore, the user may not smoothly adjust the orientation of the tube with respect to the radiographic imaging device.

The present invention has been conceived in view of the above-mentioned problem. An aim of the present invention is to smoothly adjust the orientation of the tube with respect to the radiographic imaging device.

### SUMMARY OF THE INVENTION

To achieve the abovementioned object, according to an aspect of the present invention, there is provided a radiographic imaging system including: an optical camera that obtains an optical image; a display that displays the optical image obtained by the optical camera; a radiation emitter that emits radiation to a radiographic imaging device that generates a radiographic image; and a hardware processor, wherein: the hardware processor calculates angle information of the radiographic imaging device in a right-left direction and an up-down direction with respect to a direction of the radiation emitted by the radiation emitter, and the hardware processor superposes predetermined information on the optical image and displays the predetermined information and the optical image on the display, the predetermined information being based on the calculated angle information of the radiographic imaging device in the right-left direction and the up-down direction.

### BRIEF DESCRIPTION OF THE DRAWINGS

The advantages and features provided by one or more embodiments of the invention will become more fully understood from the detailed description given hereinafter and the appended drawings which are given by way of illustration only, and thus are not intended as a definition of the limits of the present invention, and wherein:

FIG. 1 is a side view illustrating an example of a radiographic imaging system according to the present embodiment;

FIG. 2 is a perspective view of a radiographic imaging device of the radiographic imaging system shown in FIG. 1;

FIG. 3 is a block diagram illustrating the radiographic imaging device of FIG. 2;

FIG. 4 is a block diagram illustrating a radiation generating device and a console included in the radiographic imaging system of FIG. 1;

FIG. 5 is a flowchart illustrating a flow of angle information preparation process;

FIG. 6A is a diagram showing an X-axis and a Y-axis of a three axis acceleration sensor provided in the radiographic imaging device;

FIG. 6B is a diagram showing an inclination of the X-axis of the three axis acceleration sensor provided in the radiographic imaging device with respect to the horizontal plane;

FIG. 6C is a diagram illustrating an inclination of the Y-axis of the three axis acceleration sensor included in the radiographic imaging device with respect to the horizontal plane;

FIG. 7A is a diagram illustrating a method of determining the orientation of the radiographic imaging device;

FIG. 7B is a diagram illustrating the method of determining the orientation of the radiographic imaging device;

FIG. 7C is a diagram illustrating the method of determining the orientation of the radiographic imaging device;

FIG. 7D is a diagram illustrating the method of determining the orientation of the radiographic imaging device;

FIG. 8A is a view for explaining the relationship between the orientation and the roll/pitch of the radiographic imaging device;

FIG. 8B is a view for explaining the relationship between the orientation and the roll/pitch of the radiographic imaging device;

FIG. 8C is a view for explaining the relationship between the orientation and the roll/pitch of the radiographic imaging device;

FIG. 8D is a view for explaining the relationship between the orientation and the roll/pitch of the radiographic imaging device;

FIG. 9 is a table illustrating a relationship between the orientation of the radiographic imaging device and the roll/pitch;

FIG. 10A is a view illustrating the arrangement of a conventional radiographic imaging device and angle information;

FIG. 10B is a view illustrating the arrangement of the conventional radiographic imaging device and angle information;

FIG. 10C is a view illustrating the arrangement of the conventional radiographic imaging device and angle information;

FIG. 11 is a flowchart illustrating a flow of display control process;

FIG. 12 is a view illustrating an example of an angle information display screen;

FIG. 13 is a view illustrating an example of the angle information display screen;

FIG. 14 is a view illustrating an example of the angle information display screen;

FIG. 15 is a view illustrating an example of the angle information display screen;

FIG. 16 is a view illustrating an example of the angle information display screen;

FIG. 17A is a plan view and a cross-sectional view of the marker;

FIG. 17B is a plan view and a cross-sectional view of the marker;

FIG. 18 is a view illustrating an example of use of the radiographic imaging device;

FIG. 19 is a diagram illustrating an example of use of the radiographic imaging device;

FIG. 20A is a diagram for explaining a method of calculating a pitch angle of the imaging device with respect to the tube; and

FIG. 20B is a diagram for explaining the method of calculating the pitch angle of the imaging device with respect to the tube.

### DETAILED DESCRIPTION

Hereinafter, embodiments of the present invention will be described with reference to the drawings. However, the technical scope of the present invention is not limited to the following embodiments and illustrated examples.

### <1. Radiographic Imaging System>

First, a schematic configuration of a radiographic imaging system (hereinafter called a system 100) according to the present embodiment will be described, based on a case where the system 100 is a medical cart.

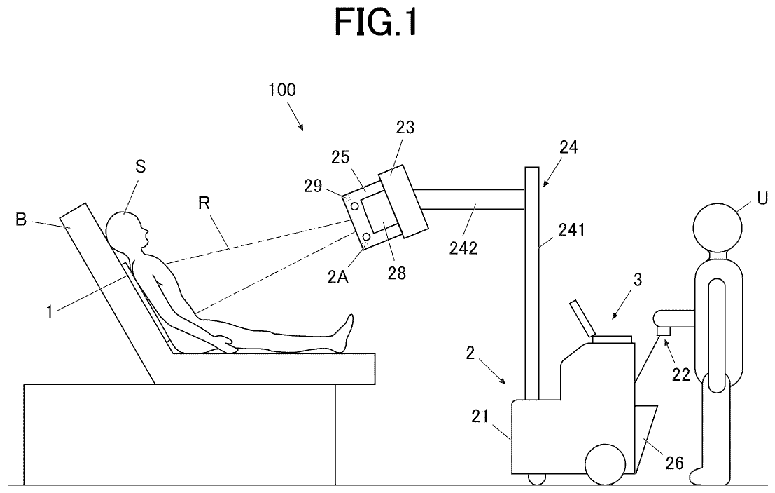

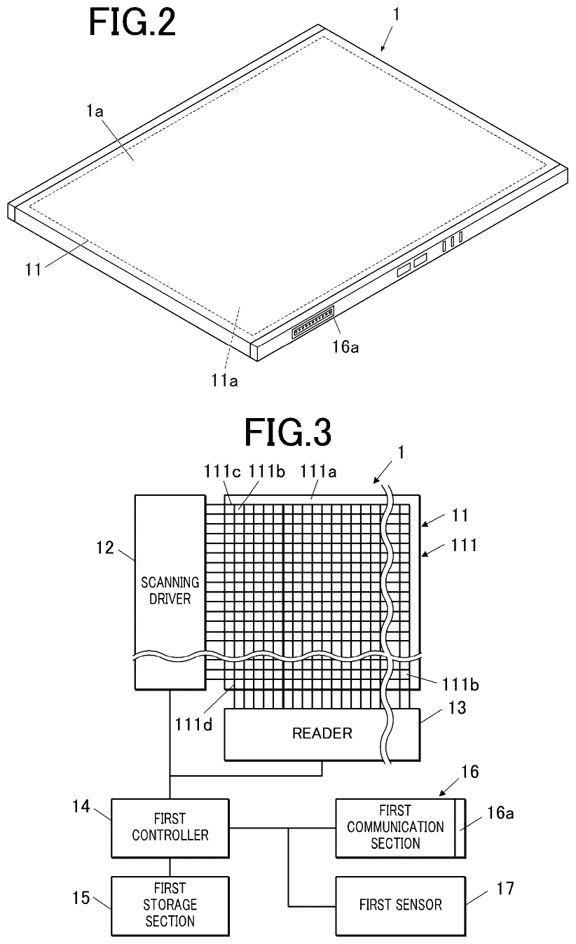

FIG. 1 is a block diagram illustrating the system 100. FIG. 2 is a perspective view illustrating a radiographic imaging device 1 included in the system 100.

As illustrated in FIG. 1, the system 100 includes, for example, the radiographic imaging device (hereinafter called the imaging device 1), a radiation generating device (hereinafter called the generating device 2), and a console 3. The devices 1 to 3 can communicate with each other via, for example, a communication network (e.g., a local area network (LAN), a wide area network (WAN), or the Internet).

The system 100 may be able to communicate with a hospital information system (HIS), a radiology information system (RIS), or the like. The system 100 may also be able to communicate with a picture archiving and communication system (PACS) and/or a dynamic analysis device. The communication network may be a wired network or a wireless network.

### [1-1. Radiographic Imaging Device]

The imaging device 1 generates a radiographic image corresponding to the radiation R received from the generating device 2. As illustrated in FIG. 1 and FIG. 2, the imaging device 1 according to the present embodiment has a panel shape and can be carried. Therefore, the imaging device 1 according to the present embodiment can be used not only by being mounted on an imaging table but also by being horizontally arranged between a subject S lying on a bed B and the bed B. Furthermore, as illustrated in FIG. 1, it is also possible to use the imaging device 1 disposed upright between the subject S in a sitting posture on the bed B part of which is up or a wheelchair and the backrest of the bed B/wheelchair. The imaging stand includes a table-like imaging stand for the supine position and an imaging stand for the standing position (wall stand).

The radiation incident surface 1a of the imaging device 1 (the surface facing the subject S) mounted on the imaging stand is parallel or orthogonal to a horizontal plane. However, in imaging without an imaging table (imaging in the bed B or the wheelchair), the radiation incident surface 1a may not be parallel or orthogonal to the horizontal plane (the radiation incident surface 1a is inclined). Further, when the imaging device 1 is interposed between a soft instrument, such as the bed B, and the subject S, the imaging device 1 may move along with the movement of the subject S. Details of the imaging device 1 will be described later.

### [1-2. Radiation Generating Device]

As illustrated in FIG. 1, the generating device 2 includes a generating device main body 21, an irradiation instruction switch 22, and a tube 23. The generating device 2 according to the present embodiment further includes a tube support portion 24, a collimator 25, and a housing 26. The generating device 2 according to the present embodiment is movable with wheels provided on the casing of the generating device 2. Details of the generating device main body 21 will be described later.

The irradiation instruction switch 22 outputs an operation signal to the generating device main body 21 in response to being operated (pressed) by the user U. Although FIG. 1 illustrates the irradiation instruction switch 22 connected to the generating device main body 21 by a wire, the irradiation instruction switch 22 may be wirelessly connected to the generating device main body 21.

When the irradiation instruction switch 22 is operated, the tube 23 generates radiation R (for example, X-rays) of a dose corresponding to a preset imaging condition in a mode corresponding to the imaging condition and emits the radiation R from the emission port.

The tube support portion 24 supports the tube 23. The tube support portion 24 according to the present embodiment includes a first support portion 241 extending upward from the generating device main body 21 to the tip end thereof; and a second support portion 242 extending forward from the upper part of the first support portion 241. The end part of the second support portion 242 supports the tube 23. The tube support portion 24 has an unillustrated joint mechanism, thereby enabling the tube 23 to be moved in the X-axis direction (the front-rear direction of the generating device 2 (the right-left direction of FIG. 1). Furthermore, since the tube support portion 24 includes the above-described joint mechanism, the tube 23 can be moved in the Y-axis direction orthogonal to the X axis (the width direction of the generating device 2 (the direction orthogonal to the plane of FIG. 1). Furthermore, since the tube support portion 24 has the above-described joint mechanism, the tube 23 can be moved in a Z-axis direction (a vertical direction (an up-down direction in FIG. 1)) orthogonal to the X axis and the Y axis. The tube support portion 24 can change the direction of the emission port of the radiation R by rotating the tube 23 on rotation axes parallel to the X axis, the Y axis, and the Z axis by a not-illustrated joint mechanism.

The collimator 25 is attached to the emission port of the tube 23 and narrows the radiation R so that the irradiation field of the radiation R emitted from the emission port has a preset rectangular shape. The collimator 25 includes a lamp button (not illustrated). When the lamp button is operated by the user, the collimator 25 emits visible light to the range corresponding to the irradiation field of the radiation R.

The housing 26 houses the imaging device 1 when the imaging device 1 is not used. The housing 26 according to the present embodiment is provided on a side surface of the generating device main body 21. The housing 26 according to the present embodiment can store multiple imaging devices 1. A connector (not illustrated) is provided in the housing 26. The connector is connected to a connector 16a (see FIG. 2) of the imaging device 1 when the imaging device 1 is stored.

### [1-3. Console]

The console 3 consists of a PC, a portable terminal, or a dedicated device. The console 3 according to the present embodiment is mounted on the generating device 2, as illustrated in FIG. 1. The console 3 can set imaging conditions to at least either the imaging device 1 or the generating device 2, based on an imaging order acquired from a different system (e.g., HIS, RIS). Herein, the imaging conditions include a tube voltage, a tube current, an irradiation time or a current-time product (mAs value), an imaging region, and an imaging direction. The console 3 can also set imaging conditions for at least either the imaging device 1 or the generating device 2, based on an operation performed on the operation part 32 by the user U (e.g., a radiologist). The console 3 can acquire image data of the radiographic image generated by the imaging device 1 and store the image data in itself or transmit the image data to a different device (e.g., a PACS, a dynamic analysis device).

### [1-4. Overview of Radiographic Imaging Using Radiographic Imaging System]

Radiographic imaging (imaging in a sitting position) using the system 100 (medical cart) configured as described above is performed as follows. First, the system 100 is arranged near the subject S (beside the bed B or the wheelchair). Next, the subject S is made to take a sitting posture. When the subject S sits on an angle-adjustable instrument (e.g., the bed B that canbe partially stood up), the angle of the backrest part is appropriately adjusted. Next, the position and orientation of the tube 23 are roughly adjusted so that the emission port of the tube 23 is directed toward the imaging target site of the subject S. Next, the imaging device 1 is taken out of the storage housing 26, and the imaging device 1 is arranged between the back of the subject S and the backrest. With reference to angle information (described in detail later), the orientation and the irradiation field of the tube 23 are finely adjusted so that the emission axis of the radiation R is orthogonal to the radiation incident surface 1a. Next, still image capturing or moving image capturing is performed (the diagnostic target site of the subject S is irradiated with the radiation R, and the imaging device 1 generates a radiographic image that shows the diagnostic target site). In the present embodiment, the still image capturing refers to capturing one image of the subject S in one imaging operation. The moving image capturing is the opposite of the still image capturing and refers to capturing a moving image in one imaging operation by continuously acquiring multiple images of the subject S. There is no restriction on whether the obtained image is displayed in real time and the length of imaging time. The moving image capturing includes dynamic imaging (also referred to as serial imaging) for acquiring multiple images of the subject S in one imaging operation. The dynamic imaging is performed by (i) repeatedly irradiating the subject S with pulsed radiation (e.g., X-rays) at predetermined time intervals (pulse irradiation) or (ii) continuously irradiating the subject S with radiation at a low dose rate without interruption (continuous irradiation). A series of images obtained by dynamic imaging is called a dynamic image. When the dynamic imaging is performed, image data of the dynamic image is transmitted to the dynamic analysis device as necessary, and the dynamics of the imaging target site (e.g., ventilation function/blood flow state of the lungs, bending and stretching of the joints) are analyzed.

The generating device main body 21 and the console 3 may be integrated (may be stored in one housing). The generating device 2 may be movable by means other than the wheels. For example, the generating device 2 may be light-weighted so that the generating device 2 can be carried by a person or mounted on a commercially available cart. For another example, the generating device 2 may have a smooth bottom surface that can slide on a floor surface. Further, either the imaging device 1 or the generating device 2 of the system 100 may be installed in an imaging room of a medical facility, for example (the other device is freely movable).

### <2. Details of Radiographic Imaging Device>

Next, details of the imaging device 1 included in the system 100 will be described. FIG. 3 is a block diagram of an electrical configuration of the imaging device 1.

### [2-1. Specific Configuration of Radiographic Imaging Device]

As illustrated in FIG. 3, the imaging device 1 includes a radiation detector 11, a scanning driver 12, a reader 13, a first controller 14, a first storage section 15, a first communication section 16, and a first sensor 17. The components 11 to 17 are electrically connected to each other.

The radiation detector 11 includes a scintillator (not illustrated) and a photoelectric conversion panel 111. The scintillator has a flat plate shape and made of columnar crystals of CsI, for example. When receiving radiation, the scintillator emits electromagnetic waves (e.g., visible light) having a wavelength longer than the wavelength of the radiation at an intensity corresponding to the dose of the received radiation (e.g., kV, mAs). The scintillator is arranged to extend parallel to the radiation incident surface 1a (see FIG. 2) of the casing.

The photoelectric conversion panel 111 is disposed to extend parallel to the scintillator on a side opposite the surface of the scintillator facing the radiation incident surface 1a. The photoelectric conversion panel 111 includes a substrate 111a and multiple charge accumulation portions 11b. The charge accumulation portions 111b are two-dimensionally arranged (e.g., in a matrix) corresponding to the pixels of the radiographic image on the surface of the substrate facing the scintillator. The charge accumulation portions 111b each include: a semiconductor element that generates an amount of charge corresponding to the intensity of the electromagnetic generated by the scintillator; and a switch element provided between the semiconductor element and the wiring connected to the reader 13. Each semiconductor element receives a bias voltage from a power supply circuit (not illustrated). Each charge accumulation portion 111b switches ON/OFF of the switch element to accumulate and discharge charges to be read out as a signal value corresponding to the received radiation.

The scanning driver 12 can switch on and off each of the switch elements by applying an on-voltage or an off-voltage to each of the scanning lines 111c in the radiation detector 11.

The reader 13 reads out, as a signal value, the amount of charge that has flowed in from the charge accumulation portions 111d via each signal line 111b of the radiation detector 11. The reader 13 may perform binning when reading out the signal values.

The first controller 14 includes a central processing unit (CPU) and a random access memory (RAM), which are not illustrated. The CPU reads various processing programs stored in the first storage section 15, loads the programs in the RAM, and executes various processes in accordance with the processing programs, thereby centrally controlling the operations of the respective units of the imaging device 1. The first controller 14 generates image data of the radiographic image, based on the signal values read by the reader 13.

The first storage section 15 consists of a hard disk drive (HDD), a semiconductor memory, or the like.

The first storage section 15 stores various programs executed by the first controller 14 and parameters and files necessary for executing the programs. The first storage section 15 may be capable of storing image data of radiographic images.

The first communication section 16 includes a communication module. The first communication section 16 can transmit and receive various signals and various data to and from other devices (e.g., the generating device 2 and the console 3) connected via wires or wirelessly over the communication network.

The first sensor 17 detects information necessary for calculating the angle information. The first sensor 17 according to the present embodiment is a three-axis acceleration sensor. The three-axis acceleration sensor detects accelerations acting in three axis (x-axis, y-axis, and z-axis) directions as information necessary for calculating the angle information and transmits the detected accelerations to the first controller 14. In a stationary state, only the gravitational acceleration acts on the three-axis acceleration sensor. Therefore, in the stationary state, the three-axis acceleration sensor detects the components of the gravitational acceleration in the three axis directions.

### [2-2. Detailed Operations of Radiographic Imaging Device]

The first controller 14 of the imaging device 1 configured as described above performs the following operation.

For example, when a predetermined condition is met, the first controller 14 causes the first sensor 17 to repeatedly detect the three axis direction components of the gravitational acceleration. Examples of the predetermined condition include, for example, (i) the imaging device 1 has been turned on, (ii) a predetermined control signal has been received from another device (the generating device 2, the console 3, or the like), and (iii) a predetermined operation has been performed on the operation part of the imaging device 1.

The first controller 14 causes the scan driver 12 to accumulate and discharge charges in the radiation detector 11 in synchronization with the timing at which the radiation R is emitted from the generating device 2. Further, the first controller 14 causes the reader 13 to read out signal values, based on charges emitted by the radiation detector 11. Further, the first controller 14 generates a radiographic image corresponding to the dose distribution of the emitted radiation R, based on the signal values read by the reader 13. In generating a still image, a radiographic image is generated only once for each press of the irradiation instruction switch 22. In generating a dynamic image, generation of a frame constituting the dynamic image is repeated by multiple times in a predetermined time (e.g., 15 times per second) for each press of the irradiation instruction switch 22. The first controller 14 transmits the image data of the generated radiographic image to other devices (e.g., the console 3, the dynamic analysis device) via the first communication section 16.

The radiation detector 11 of the imaging device 1 may not include a scintillator and may directly generate charges when the semiconductor elements receive radiation. The imaging device 1 may display the generated dynamic image in real time on a display connected to the imaging device 1 (e.g., through fluoroscopy) instead of forming image data of the dynamic image.

Even when the radiation incident surface 1a of the first sensor 17 (three axis acceleration sensor) of the imaging device 1 is parallel to the ideal horizontal plane, the output value may indicate a slight inclination. This is due to the influence of the state of the radiation detector 11 mounted on the substrate lIla, the state of the radiation detector 11 in the imaging device 1, the distortion of the casing of the imaging device 1, and so forth. Further, if the imaging device 1 receives an impact (e.g., the imaging device 1 is dropped) while being carried, the output values may indicate the inclination, or the degree of the above influence may change. Therefore, the first controller 14 may correct (calibrate) the detection value of the first sensor 17 to be output to the generating device 2. Specifically, the first controller 14 corrects the output value to indicate no inclination when the imaging device 1 is placed on an ideal horizontal plane. For another example, when the imaging device 1 is housed in a place the inclination angle of which is known with respect to the ideal horizontal plane (e.g., in the housing 26 of the medical cart), the first controller 14 corrects the output value so as to indicate that the imaging device 1 is inclined at the known inclination angle. Next, the first controller 14 stores the corrected data obtained by the correction in the first storage section 15. The correction is performed, for example, (i) at the time of initial installation of the imaging device 1 and (ii) when no corrected data is stored in the first storage section 15 of the imaging device 1 after the imaging device 1 receives an impact.

The first controller 14 may automatically correct the output values when detecting that the imaging device 1 has been stored in the housing 26. The first controller 14 may suggest the user U to make a correction (e.g., display a message suggesting the user U to make a correction). In such a case, the first controller 14 may suggest the correction only when determining that the deviation of the calculated angle information from a specific value of the rotation angle with respect to the horizontal plane when the imaging device 1 is housed in the housing 26 is greater than an allowable range.

### <3. Details of Radiation Generating Device and Console>

Next, details of the generating device 2 and the console 3 included in the system 100 will be described.

### [3-1. Specific Configuration of Radiation Generating Device]

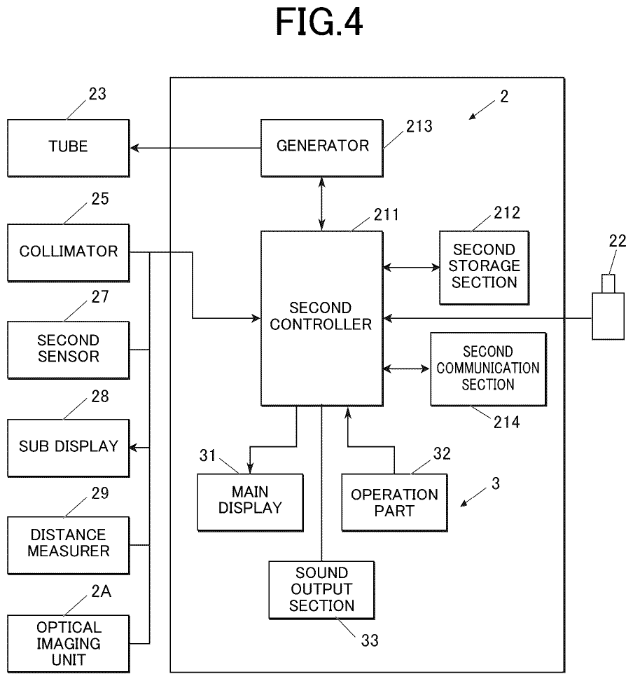

As shown in FIG. 4, the generating device 2 includes a second sensor 27, a sub display 28, a distance measurer 29, and an optical imaging unit (optical camera) 2A in addition to the generating device main body 21, the irradiation instruction switch 22, the tube 23, the tube support portion 24, the collimator 25, and the housing 26. Further, the generating device main body 21 of the generating device 2 includes a second controller 211 (hardware processor), a second storage section 212, a generator 213, and a second communication section 214.

The second sensor 27 according to the present embodiment is a three-axis acceleration sensor similar to the first sensor 17. The second sensor 27 may be a six-axis sensor or a nine axis sensor. The second sensor 27 may be of a different type from the first sensor 17.

The sub display 28 includes a monitor, such as an LCD (Liquid Crystal Display) or a CRT (Cathode Ray Tube). The sub display 28 displays various images, various kinds of information, and so forth according to instructions of display signals input by the second controller 211. The sub display 28 according to the present embodiment is provided in the casing of the collimator 25. The sub display 28 may be provided at the casing of the tube 23 or at the tube support portion 24.

The distance measurer 29 measures the SID or the SSD. The SID (source image distance) is the distance between the focal point of the radiation R and the imaging surface 11a of the imaging device 1 (the surface on which the charge accumulation portions 111b of the radiation detector 11 are provided). The SSD (source skin distance) is the distance between the focal point of the radiation R and the body surface of the subject. The SSD is substantially equal to a difference between the SID and the body thickness of the subject S. The distance measurer 29 according to the present embodiment is provided to the collimator 25.

The distance measurer 29 is a depth camera that includes: a light emitting means that emits laser light; a detecting means that detects reflected laser light; and a calculating means that calculates the distance between the light emitting means to the reflection point, based on the time from the emission of the laser light to the detection of the reflected laser light. The distance measurer 29 may include: an optical camera that generates an optical image of the imaging device 1 placed in the irradiation direction; and a calculation means that calculates the SID, based on the optical image of the imaging device 1 generated by the optical camera and the size information of the imaging device 1. The distance measurer 29 may be constituted by the combination thereof. Since the laser light is reflected on the body surface of the subject S, the distance measured by the distance measurer 29 using the laser light is often the SSD. In this case, the total of the measured SSD and the body thickness of the subject S is set as the SID. The body thickness may be a predetermined reference value, a numerical value input by the user, or an automatically calculated value from information of the subject S.

The optical imaging unit 2A includes an optical system, such as a lens, and an imaging element, such as a charge coupled device (CCD) or a complementary metal oxide semiconductor (CMOS). Under the control of the second controller 211, the optical imaging unit 2A optically images the subject S with visible light to generate optical image data and outputs the optical image data to the second controller 211. For example, the optical imaging unit 2A optically images the subject S to generate optical image data of a static image or a dynamic image (e.g., a live image).

The second controller 211 includes a CPU and a RAM. The CPU of the second controller 211 reads various programs stored in the second storage section 212, loads the programs in the RAM, executes various processes according to the developed programs, and centrally controls the operation of each section of the generating device 2.

The second storage section 212 includes a nonvolatile memory and a hard disk. The second storage section 212 stores various programs to be executed by the second controller 211 and parameters and files necessary for executing the programs.

When receiving the imaging instruction signal from the second controller 211, the generator 213 applies a voltage corresponding to preset imaging conditions to the tube 23 and applies a current corresponding to the imaging conditions to the tube 23.

The second communication section 214 includes a communication module. The second communication section 214 can transmit and receive various signals and various data to and from other devices (e.g., the imaging device 1, the console 3) connected via wires or wirelessly over the communication network.

### [3-2. Detailed Configuration of Console]

The console 3 includes a controller, a storage section, a communication section, a main display 31, an operation part 32, and a sound output section 33. The second controller 211, the second storage section 212, and the second communication section 214 of the generating device 2 also serve as the controller, the storage section, and the communication section, respectively, of the console 3 according to the present embodiment. The console 3 may include a dedicated controller, storage section, and communication section.

The main display 31 consists of a monitor, such as an LCD or a CRT.

The main display 31 displays various images and various pieces of information according to an instruction of a display signal input by the second controller 211.

The operation part 32 is operable by the user. The operation part 32 includes, for example, a keyboard (e.g., cursor keys, number input keys, and various function keys), a pointing device (e.g., a mouse), and a touch screen stacked on the surface of the main display 31. The operation part 32 outputs, to the second controller 211, a control signal corresponding to an operation performed by the user.

The sound output section 33 includes an amplifier and a speaker and outputs sound in accordance with sound information input by the second controller 211. For example, the sound output section 33 outputs a synthesized voice of a message as imaging assist information which is information for assisting the user U with radiographic imaging.

### [3-3. Detailed Operation of Radiographic Imaging System]

The second controller 211 of the generating device 2 (console 3) as described above operates as follows.

### [3-3-1. Angle Information Preparation Process]

The second controller 211 starts or resumes, for example, the angle information preparation process as illustrated in FIG. 5, in response to a predetermined condition being met. Examples of the predetermined condition for starting the angle information preparation process include, for example, (i) the generating device 2 has been turned on, (ii) the generating device 2 becomes communicable with the imaging device 1, and (iii) a predetermined operation has been performed on the operation part 32 of the console 3. Examples of the predetermined condition for resuming the angle information preparation process include, for example, (i) the emission of the radiation R has been completed and (ii) an imaging order has been selected on the operation part 32 of the console 3.

When the angle information preparation process starts, first, the second controller 211 determines whether the imaging device 1 includes the first sensor 17 (step S1). Specifically, the second controller 211 determines whether the imaging device 1 includes the first sensor 17 by referring to information on the presence/absence of the first sensor 17 stored in the imaging device 1. The second controller 211 may refer to the imaging device ID stored in the imaging device 1 and comparison information between the imaging device 1 and the presence/absence of the first sensor 17 stored in a different device (e.g., the console 3).

When determining in step S1 that the imaging device 1 does not include the first sensor 17 (step S1: NO), the second controller 211 ends the angle information preparation process (the second controller 211 does not permit display of the angle information of the imaging device 1 relative to the tube 23). That is, the display of the angle information of the imaging device 1 relative to the tube 23 is not permitted. Thus, when an imaging device that does not have the first sensor 17 is used, the angle information of the imaging device relative to the tube 23 is not displayed. This prevents the user U from making a mistake.

When determining in step S1 that the imaging device 1 includes the first sensor 17 (Step S1: YES), the second controller 211 acquires gravitational acceleration information from the first sensor 17 (step S2). The second controller 211 also acquires gravitational acceleration information from the second sensor 27 (step S2). Herein, the gravitational acceleration information acquired from the first sensor 17 is information indicating three axis direction components of the gravitational acceleration detected by the first sensor 17. The gravitational acceleration information acquired from the second sensor 27 is information indicating three axis direction components of the gravitational acceleration detected by the second sensor 27.

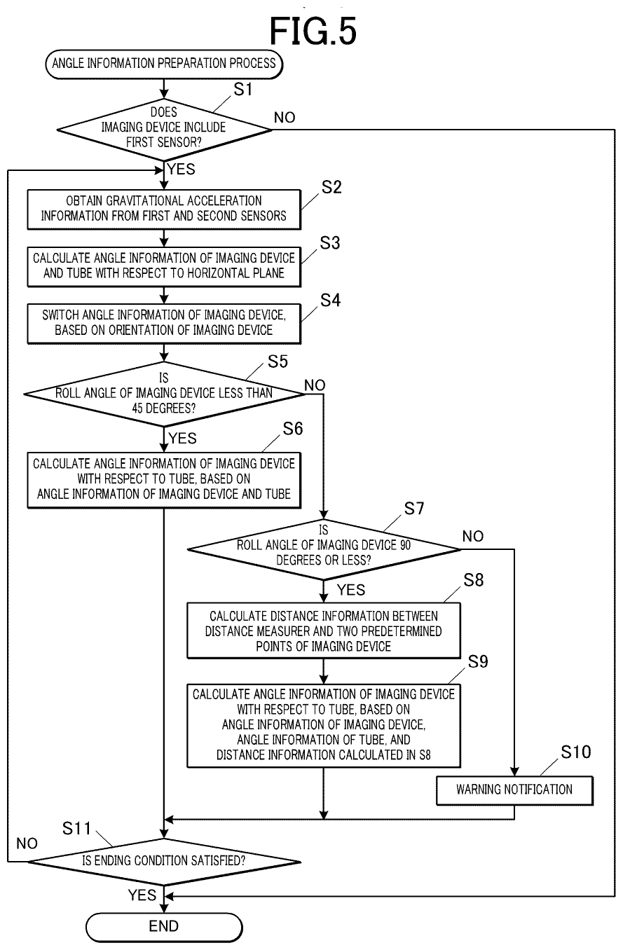

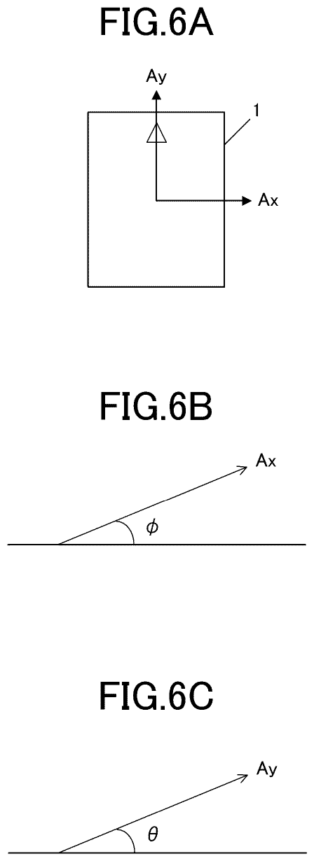

Next, the second controller 211 calculates angle information of each of the imaging device 1 and the tube 23 with respect to the horizontal plane (step S3). To be specific, as illustrated in FIG. 6B, the second controller 211 calculates, as the angle information of the imaging device 1, an inclination φ (a pitch angle) of the X-axis (Ax in FIG. 6A) of the three-axis acceleration sensor (the first sensor 17) with respect to the horizontal plane by using the following Expression (1). Hereinafter, the inclination φ of the X-axis (Ax in FIG. 6A) of the three-axis acceleration sensor (the first sensor 17) with respect to the horizontal plane may be referred to as a pitch angle before switching. Further, as illustrated in FIG. 6C, the second controller 211 calculates, as the angle information on the imaging device 1, an inclination θ (a roll angle) of the Y-axis (Ay in FIG. 6A) of the three axis acceleration sensor (the first sensor 17) with respect to the horizontal plane by using the following Expression (2). Hereinafter, the inclination θ of the Y-axis (Ay in FIG. 6A) of the three axis acceleration sensor (the first sensor 17) with respect to the horizontal plane may be referred to as a roll angle before switching. Further, the second controller 211 calculates angle information of the tube 23 in the same manner.

$$\begin{matrix}[ Expression ] & \\ ∅ = a tan ( Ax / \sqrt{{Ay}^{2} + {Az}^{2}} ) \times 180 / π & ( 1 )\end{matrix}$$

$$θ = a tan ( Ay / \sqrt{{Ax}^{2} + {Az}^{2}} ) \times 180 / π \qquad ( 2 )$$

Ax, Ay, and Az are components of gravitational acceleration in the X, Y, and Z directions, respectively.

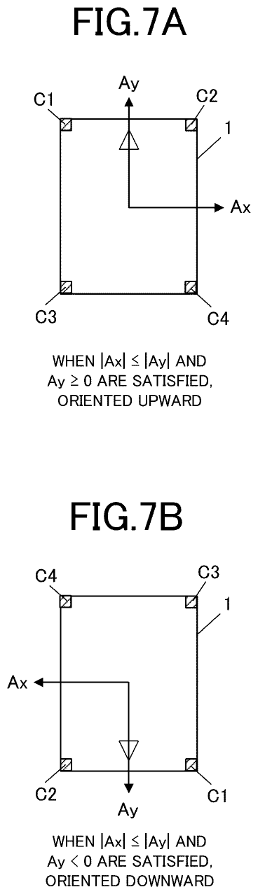

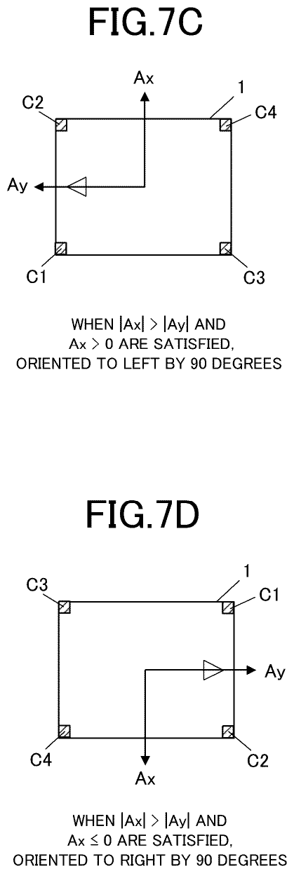

Next, the second controller 211 switches the angle information of the imaging device 1 according to the direction of the imaging device 1 (step S4). Specifically, first, the second controller 211 determines the orientation of the imaging device 1 from the gravitational acceleration output by the three-axis acceleration sensor (the first sensor 17). For example, when |Ax|≤|Ay| and Ay≥0 are satisfied, the second controller 211 determines that the imaging device 1 is oriented upward. Herein, as illustrated in FIG. 7A, “upward” means a state where the imaging device 1 is vertically oriented and the Δ mark of the imaging device 1 is directed upward. The second controller 211 determines that the imaging device 1 is oriented downward when |Ax|≤Ay| and Ay<0 are satisfied. Herein, as illustrated in FIG. 7B, “downward” means a state where the imaging device 1 is vertically oriented and the Δ mark of the imaging device 1 is directed downward. Further, when |Ax|>Ay| and Ax>0 are satisfied, the second controller 211 determines that the imaging device 1 is oriented to the left by 90 degrees. Herein, as illustrated in FIG. 7C, 90 degrees to the left means a state where the imaging device 1 is in the lateral direction and the Δ mark of the imaging device 1 is directed to the left. When |Ax|>|Ay| and Ax≤0 are satisfied, the second controller 211 determines that the imaging device 1 is oriented to the right by 90 degrees. Herein, as illustrated in FIG. 7D, 90 degrees to the right means a state where the imaging device 1 is in the lateral direction and the Δ mark of the imaging device 1 is directed to the right. The second controller 211 may determine the orientation of the imaging device 1, based on the image of the imaging device 1 captured by the optical imaging unit 2A.

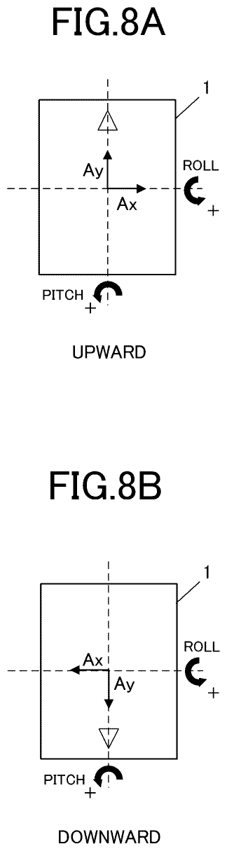

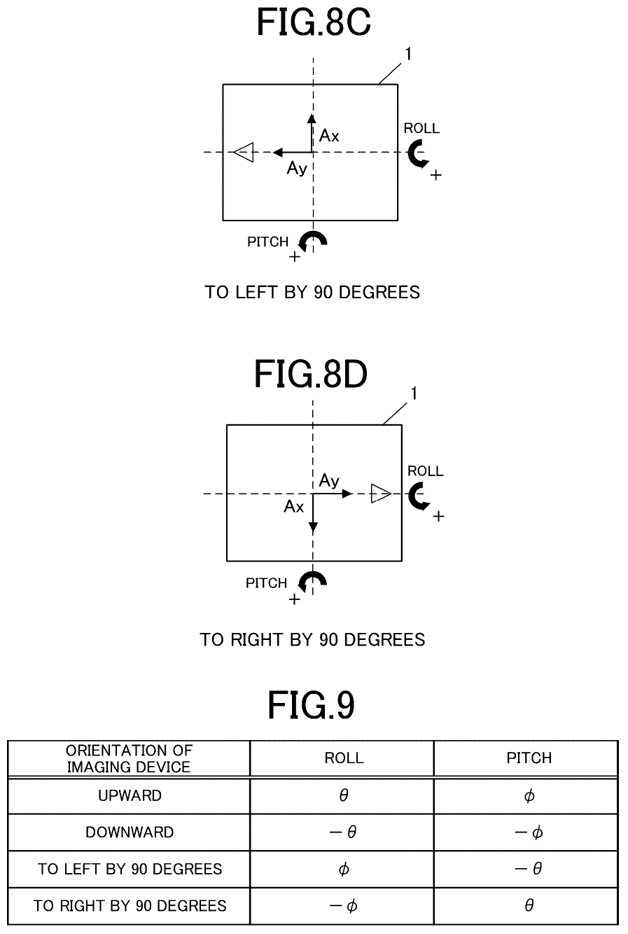

Next, as illustrated in FIG. 8A and FIG. 9, when the imaging device 1 is oriented upward, the second controller 211 outputs the inclination θ of the Y axis with respect to the horizontal plane calculated in step S3 (i.e., the roll angle before switching) as the roll angle after switching. Further, the second controller 211 outputs the inclination φ of the X axis with respect to the horizontal plane (i.e., the pitch angle before switching) as the pitch angle after switching. Herein, the roll angle and the pitch angle after switching are a roll angle and a pitch angle that do not depend on the X axis and the Y axis (Ax, Ay in FIG. 6A) of the three-axis acceleration sensor (the first sensor 17). The roll angle and the pitch angle before switching are a roll angle and a pitch angle whose definitions do not change depending on the direction of the imaging device 1. The roll angle and the pitch angle after switching are a roll angle and a pitch angle corresponding to the direction of the imaging device 1. Further, as illustrated in FIG. 8B and FIG. 9, when the imaging device 1 is oriented downward, the second controller 211 outputs the inclination −θ of the Y axis with respect to the horizontal plane calculated in step S3 (the roll angle before switching) as the roll angle after switching. Further, the second controller 211 outputs the inclination −φ of the X axis with respect to the horizontal plane (i.e., the pitch angle before switching) as the pitch angle after switching. As illustrated in FIG. 8C and FIG. 9, when the imaging device 1 is oriented to the left by 90 degrees, the second controller 211 outputs the inclination φ of the X axis calculated in step S3 with respect to the horizontal plane (i.e., the pitch angle before switching) as the roll angle after switching. Further, the second controller 211 outputs the inclination −θ of the Y axis with respect to the horizontal plane (the roll angle before switching) as the pitch angle after switching. As illustrated in FIG. 8D and FIG. 9, when the orientation of the imaging device 1 is oriented to the right by 90 degrees, the second controller 211 outputs the inclination −φ of the X axis with respect to the horizontal plane calculated in step S3 (i.e., the pitch angle before switching) as the roll angle after switching. Further, the second controller 211 outputs the inclination θ of the Y axis with respect to the horizontal plane (i.e., the roll angle before switching) as the pitch angle after switching.

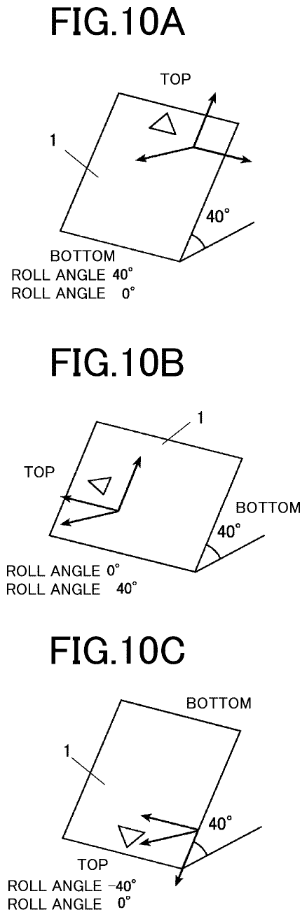

In a case where the size of the imaging device 1 to be used is 14 inches×17 inches, the imaging device 1 is normally used in a state as illustrated in FIG. 10A. However, depending on the physique of the subject S, the imaging device 1 may be rotated by 90 degrees (90 degrees to the left) to perform imaging, as illustrated in FIG. 10B. Since the imaging device 1 has a simple panel shape, the user U may perform imaging while keeping the imaging device 1 upside down (rotating the imaging device 1 by 180°) as illustrated in FIG. 10C without considering the up and down of the imaging device 1. In such a case, the generated radiographic image is also upside down but can be rotated later. Therefore, when the imaging device 1 is used while being rotated as described above, the relationship between the roll angle and the pitch angle is reversed, or the roll angle or the pitch angle has a negative value. In such a case, a problem may occur when the roll angle and the pitch angle of the tube 23 are finely adjusted. Therefore, in step S4, the second controller 211 switches the angle information of the imaging device 1 according to the direction of the imaging device 1. That is, the second controller 211 outputs the roll angle and the pitch angle after switching to prevent the above-described problem. Therefore, it can be said that the roll angle and the pitch angle before switching are the roll angle and the pitch angle before matching the definitions of the roll angle and the pitch angle of the imaging device 1 with the definitions of the roll angle and the pitch angle of the generating device 2 (tube 23). Further, it can be said that the roll angle and the pitch angle after switching are the roll angle and the pitch angle after matching the definitions of the roll angle and the pitch angle of the imaging device 1 with the definitions of the roll angle and the pitch angle of the generating device 2 (tube 23).

The second controller 211 detects whether the imaging device 1 faces the irradiation surface or the non-irradiation surface, based on the gravitational acceleration information of the Z axis of the three-axis acceleration sensor. When the imaging device 1 faces the non-irradiation surface, the second controller 211 may stop the above-described process in step S4 and draw the user U's attention (e.g., display a warning). After the imaging device 1 is placed under or behind the subject S, the use U cannot notice whether the imaging device 1 is inside out until the imaging ends. According to the present embodiment, even when the imaging device 1 is disposed under or behind the subject S, the use U is allowed to check whether the imaging device 1 is turned inside out. Thus, the user U can prevent failure in imaging and avoid unnecessarily exposing the subject S to radiation.

Returning to FIG. 5, the second controller 211 determines whether the roll angle of the imaging device 1 after switching in step S4 (roll angle after switching, angle information) is less than 45 degrees (step S5). That is, the second controller 211 determines whether the pitch angle of the imaging device 1 after switching in step S4 (pitch angle after switching; angle information) is available.

In step S5, when determining that the roll angle of the imaging device 1 (the roll angle after switching) is less than 45 degrees (step S5: YES), the second controller 211 calculates angle information of the imaging device 1 with respect to the tube 23, based on the angle information of the imaging device 1 and the tube 23 (step S6). Herein, the angle information of the imaging device 1 with respect to the tube 23 is information on the angle of the imaging device 1 in the right-left direction and the up-down direction (i.e., in the roll direction and the pitch direction) with respect to the irradiation direction of the radiation R emitted from the tube 23 (radiation emitter). To be specific, the second controller 211 calculates a difference between the roll angle of the imaging device 1 after switching in step S4 (roll angle after switching) and the roll angle of the tube 23 calculated in step S3 (angle information); and the second controller 211 determines the difference as the roll angle of the imaging device 1 with respect to the tube 23 (angle information). The second controller 211 also calculates a difference between the pitch angle of the imaging device 1 after switching in step S4 (pitch angle after switching) and the pitch angle of the tube 23 calculated in step S3 (angle information); and the second controller 211 determines the difference as the pitch angle of the imaging device 1 with respect to the tube 23 (angle information). Hereinafter, the method of calculating the angle information of the imaging device 1 with respect to the tube 23 in step S6 may be referred to as a first angle information calculation method.

Next, the second controller 211 determines whether at least one of the following ending conditions (1) and (2) has been satisfied (step S11).
(1) The irradiation instruction switch 22 has been operated
(2) The irradiation of the radiation R has started

Since the radiation image to be generated is determined at the time the radiation R is emitted, there is low necessity of continuing the angle information preparation process and the display control process (described later). In particular, in dynamic imaging, radiographic images are often acquired while a patient performs a predetermined action, such as a breathing action or a bending motion of a joint. Although the imaging device 1 may vibrate or the inclination thereof may change owing to these actions, imaging may not be stopped owing to such vibrations or changes. If the result of a state determination process (described later) is displayed during imaging, the user U may erroneously stop the dynamic imaging halfway, based on the determination result. Therefore, in particular in dynamic imaging, differentiating the display of angle information before starting irradiation (during non-irradiation) and after starting irradiation (during irradiation) is beneficial in preventing imaging failure.

Depending on the type of dynamic imaging, the patient may be kept still from the start to the end of irradiation (from the start to the end of one time of dynamic imaging). For example, in dynamic imaging for observing the blood flow state of the lungs, in order to observe a minute change of the lung field owing to the blood flow of the lungs that changes due to the heartbeat, the patient is instructed to hold his/her breath and not to move his/her body during imaging. In such imaging, a change in angle information during imaging is important in determining whether to stop imaging. Therefore, the ending condition may be “the irradiation of the radiation R has ended, in other words, one time of imaging (dynamic imaging) has ended” instead of the above-mentioned ending condition (2): The irradiation of the radiation R has started.

When determining in step S11 that the end condition is not satisfied (step S11: NO), the second controller 211 returns to step S2 and repeats the subsequent processes. That is, the second controller 211 repeats calculation of the angle information (roll angle and pitch angle) of the imaging device 1 relative to the tube 23 until the ending condition is satisfied.

When determining in step S11 that the ending condition is satisfied (step S11: YES), the second controller 211 ends the angle information preparation process.

In Step S5, when determining that the roll angle after switching of the imaging device 1 is not less than 45 degrees (i.e., the roll angle after switching of the imaging device 1 is equal to or greater than 45 degrees) (Step S5: NO), the second controller 211 determines whether or not the roll angle after switching is equal to or less than 90 degrees (Step S7).

When determining in step S7 that the roll angle of the image sensing device 1 after switching is 90 degrees or less (step S7: YES), the second controller 211 measures the distance between the tube 23 and the imaging device 1 by using the distance measurer 29 and calculates (outputs) distance information. To be specific, the second controller 211 calculates, using the distance measurer 29, information on the distances from the distance measurer 29 provided to the tube 23 to two predetermined points on the imaging device 1 (step S8). More specifically, when the imaging device 1 is oriented upward, the second controller 211 calculates (i) distance information from the distance measurer 29 to the corner C1 and (ii) distance information from the distance measurer 29 to the corner C2 among the four corners C1 to C4 of the imaging device 1, as illustrated in FIG. 7A. Herein, in a case where the imaging device 1 is oriented upward, the corner C1 and the corner C2 correspond to two left and right corners on the top side of the imaging device 1. When the imaging device 1 is oriented upward, the second controller 211 may calculate (i) distance information from the distance measurer 29 to the corner C3 and (ii) distance information from the distance measurer 29 to the corner C4. When the imaging device 1 is oriented upward, the corner C3 and the corner C4 correspond to two left and right corners on the bottom side of the imaging device 1. When the imaging device 1 is oriented downward, the second controller 211 calculates (i) distance information from the distance measurer 29 to the corner C3 and (ii) distance information from the distance measurer 29 to the corner C4 among the four corners C₁ to C₄ of the imaging device 1, as illustrated in FIG. 7B. Herein, when the imaging device 1 is oriented downward, the corner C3 and the corner C4 correspond to two right and left corners on the top side of the imaging device 1. When the imaging device 1 is oriented downward, the second controller 211 may calculate (i) distance information from the distance measurer 29 to the corner C1 and (ii) distance information from the distance measurer 29 to the corner C2. When the imaging device 1 is oriented downward, the corner C1 and the corner C2 correspond to two right and left corners on the bottom side of the imaging device 1. Further, when the imaging device 1 is oriented to the left by 90 degrees, the second controller 211 calculates (i) distance information from the distance measurer 29 to the corner C2 and (ii) distance information from the distance measurer 29 to the corner C4 among the four corners C₁ to C₄ of the imaging device 1, as illustrated in FIG. 7C. Here, when the imaging device 1 is oriented to the left by 90 degrees, the corner C2 and the corner C4 correspond to two right and left corners on the top side of the imaging device 1. When the imaging device 1 is oriented to the left by 90 degrees, the second controller 211 may calculate (i) distance information from the distance measurer 29 to the corner C1 and (ii) distance information from the distance measurer 29 to the corner C3. When the imaging device 1 is oriented to the left by 90 degrees, the corner C1 and the corner C3 correspond to two right and left corners on the bottom side of the imaging device 1. Further, when the imaging device 1 is oriented to the right by 90 degrees, the second controller 211 calculates (i) distance information from the distance measurer 29 to the corner C1 and (ii) distance information from the distance measurer 29 to the corner C3 among the four corners C1 to C4 of the imaging device 1, as illustrated in FIG. 7D. Here, when the imaging device 1 is oriented to the right by 90 degrees, the corner C1 and the corner C3 correspond to two right and left corners on the top side of the imaging device 1. When the imaging device 1 is oriented to the right by 90 degrees, the second controller 211 may calculate (i) distance information from the distance measurer 29 to the corner C2 and (ii) distance information from the distance measurer 29 to the corner C4. When the imaging device 1 is oriented to the right by 90 degrees, the corner C2 and the corner C4 correspond to two right and left corners on the bottom side of the imaging device 1. The distance between the tube 23 and the imaging device 1 may be measured using the optical imaging unit 2A provided to the tube 23. Here, when the above-described measurement is performed using the distance measurer 29 or the optical imaging unit 2A, the definition of the distance to be measured varies according to the grade or specifications of the radiographic imaging system, so that the number of man-hours for development increases. To deal with this, the distance to be measured may be converted into a distance from the focal position of the radiation R in the tube 23. Thus, the subsequent processing can be made common regardless of the grade or specification of the radiographic imaging system, so that the development cost for producing the radiographic imaging system can be reduced.

In radiographic imaging (in particular, dynamic imaging) in the present embodiment, a direct radiation region (through region) in which the subject S is not imaged is provided in imaging in order to suppress the influence of fluctuations in radiation on dynamic analysis. The above-described distance information is calculated by using the direct radiation region. That is, the above-described four corners C₁ to C₄ of the imaging device 1 are used as the direct radiation regions (through regions). In this case, the console 3 may correct the radiographic image, based on the pixel values of the direct radiation regions (through region). That is, the console 3 corrects the radiographic image, based on the pixel values of the regions used for measuring the distance between the radiation emitter and the radiographic imaging device in the irradiation surface of the radiographic imaging device. In the moving image capturing or the dynamic image capturing, the console 3 may correct each frame of the radiographic image, based on the pixel values of the direct radiation region (through region) of the frame. Herein, the direct radiation region (through region) is recognized by identifying differences in geometric features of the four corners C1 to C4 in the images. Therefore, the four corners C1 to C4 are formed to have different geometric features. For example, on the irradiation surface of the imaging device 1, a framing that indicates an area in which X-rays can be detected is drawn. The shapes of the four corners of the framing are made different, such as “F”, “+”, and “T”. All of the four corners C1 to C4 may have different shapes. For another example, two shapes of “+” and “T” may be used such that the upper left and lower left have “+” and the upper right and the lower right have “T”. Thus, based on the combinations of the shapes of adjacent two corners, each side can be identified, and accordingly, the four corners can be identified. For example, if the left of a side is “+” and the right of the side is “T”, the side can be determined to be the upper side. If both the left and right of a side are “T”, the side can be determined as the right side. Thus, it is sufficient that the shapes of the four corners have at least two different geometric features. The geometric feature provided to each of the four corners C1 to C4 is within an 8 cm square. The smaller the size of the geometric feature is, the easier it is for the user to secure the direct radiation region (through region) and to perform positioning. Specifically, it is preferable that the geometric feature at each of the four corners has the size of 5 cm square rather than 8 cm square. More preferably, the geometric feature at each of the four corners has the size of 3 cm square. Furthermore, stickers having different geometric characteristics (marks) may be attached to the four corners C1 to C4 (e.g., within the size of 8 cm square), and the stickers may be recognized. Thus, even when the panel is hidden by the grid in imaging, it is possible to cope with this by attaching a seal on the grid.

The parts for calculating the distance information are not limited to the four corners C1 to C4 of the imaging device 1, as long as the parts are two parts at different positions in the horizontal direction (right-left direction). Further, the parts for calculating the distance information may be changed according to the imaging conditions. Further, in calculating the distance information, the second controller 211 may determine whether the direct irradiation region (through region) is secured by using the optical imaging unit 2A or the distance measurer (depth camera) 29. The second controller 211 may display the determination result on the sub display 28 or output the sound indicating the determination result from the sound output section 33 to prevent failure of radiographic imaging. When determining that the direct radiation region (through region) is secured, the second controller 211 may display information (guide information) regarding the expected direct radiation region on the sub display 28. Further, the determination result may be used to determine the reliability of the distance information calculated by the distance measurer 29 or the pitch angle (angle information) of the imaging device 1 calculated based on the distance information. Based on the reliability determination, if the reliability is low, the display of the angle information of the imaging device 1 with respect to the tube 23 may not be permitted, or the display of the angle information may be performed in a manner different from a normal case where the reliability is high (e.g., the information is grayed out). When the reliability is low, the reliability information may be reflected on various controls of the system 100 such that the irradiation of the radiation R is not permitted or an alert display is performed, for example. Further, the above-described distance information may not be calculated using the distance measurer 29 but may be calculated using the optical imaging unit 2A, for example. Specifically, the optical imaging unit 2A captures an image of a structure or mark near the irradiation surface in the direct radiation region (through region). Based on the geometric distortion of the imaged structure or mark, the above-described distance information is calculated. Herein, it is preferable that the information on the structure or the mark is associated with information on the imaging order.

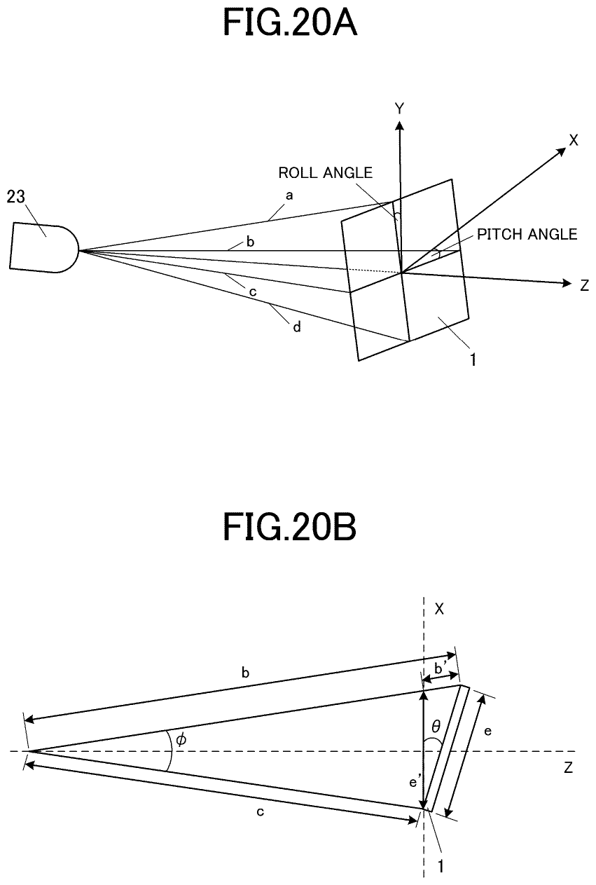

Referring back to FIG. 5, the second controller 211 calculates angle information of the imaging device 1 with respect to the tube 23, based on the angle information of the imaging device 1 and the tube 23 and the distance information calculated in step S8 (step S9). Herein, the angle information of the imaging device 1 with respect to the tube 23 is information on the angle of the imaging device 1 in the right-left direction and the up-down direction (i.e., in the roll direction and the pitch direction) with respect to the irradiation direction of the radiation emitted from the tube 23 (radiation emitter). To be specific, the second controller 211 calculates a difference between the roll angle of the imaging device 1 after switching in step S4 (roll angle after switching) and the roll angle of the tube 23 calculated in step S3 (angle information); and the second controller 211 determines the difference as the roll angle of the imaging device 1 with respect to the tube 23 (angle information). The second controller 211 also calculates the pitch angle (angle information) of the imaging device 1 with respect to the tube 23, based on the distance information from the range distance measurer 29 to the two predetermined points of the imaging device 1 calculated in step S8. Specifically, the pitch angle (cos 0: see FIG. 20A and FIG. 20B) of the imaging device 1 with respect to the tube 23 can be calculated by the following expression (a). In the expression (a), “e” can be calculated by the following expression (b). Further, in the formula (b), “cos 4D” can be calculated by the following expression (c). Further, in the formula (a), “b” can be calculated by the following expression (d). FIG. 20A and FIG. 20B are diagrams illustrating the positional relationships between the tube 23 (distance measurer 29) and two predetermined points on the imaging device 1. The Z axis in the figures indicates the irradiation axis (optical axis) of the radiation R. The X axis and the Y axis in the figures are on a plane perpendicular to the Z axis and intersect each other at a right angle at the intersection of the Z axis and the plane. The Y axis in the figures extends in the top-bottom direction of the rectangular irradiation field of the irradiation beam R. When the irradiation field R is emitted in the horizontal direction and the upper side of the rectangular irradiation field is horizontal, the Y axis is in the vertical direction. The X axis extends in the right-left direction of the rectangular irradiation field of the radiation R. When the irradiation field R is emitted in the horizontal direction and the upper side of the rectangular irradiation field is horizontal, the X axis is in the horizontal direction. In figures, “b” indicates the distance from the tube 23 (the distance measurer 29) to one of the two predetermined points of the imaging device 1. In figures, “c” indicates the distance from the tube 23 (the distance measurer 29) to the other of the two predetermined points of the imaging device 1. That is, “b” and “c” in the figures are the distance information from the tube 23 (the distance measurer 29) to the two predetermined points of the imaging device 1. Further, “e” in the figures represents the distance between the two predetermined points of the imaging device 1. Hereinafter, the method of calculating the angle information of the imaging device 1 with respect to the tube 23 in step S9 may be referred to as a second angle information calculation method.

$$cos θ = ( {e}^{2} + {e}^{′2} - {b}^{′2} ) / 2 e \cdot {e}^{′} \qquad ( a )$$

$${e}^{′} = {( {b}^{2} + {c}^{2} - 2 b \cdot c \cdot cos Φ )}^{1 / 2} \qquad ( b )$$

$$cos Φ = ( {b}^{2} + {c}^{2} - {e}^{2} ) / 2 b \cdot c \qquad ( c )$$

$${b}^{′} = b - c \qquad ( d )$$

Next, the second controller 211 determines whether the above-described ending condition has been satisfied (step S11). When determining in step S1I that the ending condition is not satisfied (step 511: NO), the second controller 211 returns to step S2 and repeats the subsequent processes. On the other hand, when determining in step S11 that the ending condition has been satisfied (step S11: YES), the second controller 211 ends the angle information preparation process.

In step S7, when determining that the roll angle after switching of the imaging device 1 is not equal to or less than 90 degrees (i.e., the roll angle after switching of the imaging device 1 exceeds 90 degrees) (step S7: NO), the second controller 211 outputs a warning to notify that the roll angle after switching exceeds 90 degrees (step S10). Specifically, the second controller 211 displays a warning message notifying that the roll angle after switching of the imaging device 1 exceeds 90 degrees on the sub display 28 or outputs the warning message (voice) from the sound output section 33. The second controller 211 then proceeds to step S1I and performs the subsequent processing. In a case where a standing position or a sitting position (not a semi reclining position) is imaged, the roll angle of the imaging device 1 after switching is close to 90 degrees and may be greater than 90 degrees depending on the inclination of the imaging device 1. Therefore, the angle for determination in step S7 (the roll angle after switching) may include a margin to 90 degrees, such as 100 degrees.

### [3-3-2. Angle Information Preparation Processing and Others]

After calculating the angle information of the imaging device 1 with respect to the tube 23 in the angle information preparation process, the second controller 211 may perform a state determination process. In the state determination process, the second controller 211 determines whether the angle information of the imaging device 1 relative to the tube 23 is within a predetermined reference range. Herein, the determination may be made only on the pitch angle. In such a case, the second controller 211 may use the determination result as the angle information of the imaging device 1 with respect to the tube 23. The second controller 211 may use the distance measurer 29 to measure the SID and the SSD. Further, when the SID and the SSD are measured by using the distance measurer 29, the second controller 211 may estimate the body thickness of the subject S from the SID and the SSD (body thickness=SID−SSD). The second controller 211 may determine whether the SID measured by the distance measurer 29 is within a predetermined reference range.

In performing the above-described state determination process, the second controller 211 may determine whether dynamic imaging is included in the imaging order before executing the state determination process. When determining that the imaging order includes dynamic imaging, the second controller 211 may change (narrow) the reference range to be used in the subsequent state determination process. This is because the alignment accuracy required in dynamic imaging, which is used for dynamic analysis, is higher than in still image capturing. When determining that the imaging order includes dynamic imaging, the second controller 211 may perform the subsequent state determination process. When determining that the imaging order includes still image capturing, the second controller 106 may not perform the subsequent state determination process. In performing the state determination process, the second controller 211 may change the reference range according to the presence or absence of the grid and the type of the grid. This is because when the grid ratio increases, the influence of the oblique incidence of the radiation R increases (variations in density occur in the radiographic image owing to the influence of the cutoff by the grid).

In the angle information preparation process, the second controller 211 performs one time of the calculation process in step S3 for multiple times of the obtaining process in step S2. In the calculation process, the second controller 211 may calculate an average value, a median value, or the like of the multiple pieces of angle information of the imaging device 1 and the tube 23 as the angle information of the imaging device 1 and the tube 23 with respect to the horizontal plane. After the imaging device 1 is arranged under or behind the subject S, the angle display may vary due to the influence of the breathing of the subject S, for example. According to the above, the variation of the angle information due to breathing may be reduced. Further, since the variation of the angle information is reduced, the direction of the tube 23 can be adjusted finely and easily.

The method of calculating the angle information of the imaging device 1 with respect to the tube 23 in the angle information preparation process is merely an example. In the above calculation method, the method of calculating the angle information of the imaging device 1 is changed, based on the posture of the imaging device 1. Specifically, the method of calculating the pitch angle (the angle information of the imaging device 1) is changed depending on the roll angle after switching of the imaging device 1. More specifically, the second controller 211 compares the value of the “roll angle after switching” derived in step S4 of the angle information preparation process with a predetermined threshold value; and based on the comparison result, the second controller 211 determines which to use as the pitch angle, the pitch angle after switching derived in step S4 of the angle information preparation process or the pitch angle derived from the distance information calculated in step S8 of the angle information preparation process. When the roll angle is less than the threshold value, both the roll angle and the pitch angle as the angle information of the imaging device 1 are calculated based on the gravitational acceleration information of the imaging device 1. When the roll angle is equal to or greater than the threshold value, the roll angle as the angle information of the imaging device 1 is calculated based on the gravitational acceleration information of the imaging device 1, whereas the pitch angle of the angle information is calculated, based on the distance information of the distance measurer 29. That is, the angle information of the radiographic imaging device (imaging device 1) is calculated, based on the distance information of the measurer (distance measurer 29) and/or the gravitational acceleration information of the radiographic imaging device. In other words, the method of calculating the angle information of the radiographic imaging device is changed (selected or determined), based on the gravitational acceleration information of the radiographic imaging device.

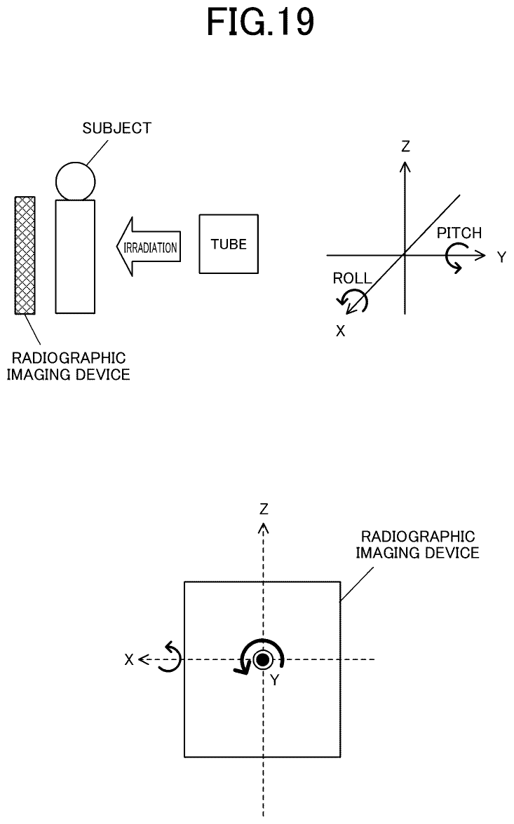

As described above, in the arrangement for horizontal irradiation as illustrated in FIG. 19, the pitch angle calculated based on the gravitational acceleration information of the radiographic imaging device (imaging device 1) does not indicate the inclination of the imaging device 1 in the right-left direction but indicates the rotation angle of the imaging device 1 on the axis perpendicular to the irradiation surface of the imaging device 1. That is, as the roll angle is closer to 90 degrees, the pitch angle that is calculated based on the gravitational acceleration information of the imaging device 1 becomes less suitable for the intended use. On the other hand, the pitch angle that is calculated based on the above-described distance information measured by the distance measurer 29 may be inaccurate if there is not a sufficient direct irradiated region. Accordingly, the pitch angle cannot be derived, or the accuracy thereof may be decreased. Since both methods of calculating the pitch angle have advantages and disadvantages, it is desirable to appropriately determine the calculation method, depending on the purpose of imaging and the scene of use.

Although the threshold value used for switching is set to 45 degrees in the above-described calculation method, the threshold may be other than 45 degrees. Among the four main imaging technique categories (types of positioning) of the spine position imaging, the semi-seated position imaging, the sitting position imaging, and the standing position imaging, the roll angle is substantially 0 degree in the spine position imaging; about 30 to 45 degrees in semi-seated position imaging; 45 to 90 degrees in the sitting position imaging; and substantially 90 degrees in the standing position imaging. When the threshold is set in the range of 10 to 25 degrees (e.g., 15 degrees), the imaging techniques are classified into the spine position imaging and the other three imaging techniques. Thus, appropriate angle information is selected for the imaging technique, and the user can use the angle information without confusion.

The method of calculating the pitch angle may be changed, based on the imaging technique included in the order information, instead of based on the roll angle. Thus, the same usability as described above can be achieved. In other words, the method of calculating the angle information of the radiographic imaging device (imaging device 1) is changed (selected or determined), based on information of imaging to be performed (imaging technique). Further, the method of calculating the angle information of the imaging device 1 may be changed, based on (i) the imaging information (imaging technique classification) and (ii) the threshold value of the roll angle. Further, the threshold value for switching may be selected according to the imaging technique classification. The above two may be combined. For example, when the imaging technique is the spine position imaging, the pitch angle may be always calculated based on the gravitational acceleration information of the imaging device 1. When the imaging technique is the standing position imaging, the pitch angle may be always calculated based on the distance information of the distance measurer 29. The position of the patient can be changed from the semi-seated position/sitting position to the spine position depending on the state of the patient. In such a case, the method of calculating the pitch angle is changed based on the threshold value of the roll angle, and the threshold value is set to 15 degrees, for example. In other words, the method of calculating the angle information of the radiographic imaging device is changed (selected or determined), based the information of imaging to be performed (imaging technique classification) and the gravitational acceleration information of the radiographic imaging device.

When imaging is performed in a ward or an ICU using a medical cart, the sitting position imaging or the standing position imaging may be rarely performed. In such a case, the threshold value may be set within the range of 50 to 75 degrees (e.g., 60 degrees). With such a threshold value, when the imaging is performed in a hospital ward or an ICU, the pitch angle calculated based on the gravitational acceleration information of the imaging device 1 is always selected; whereas when the standing position imaging is performed by bringing the medical cart into the imaging room, the pitch angle calculated based on the distance information of the distance measurer 29 is selected. Thus, angle information is appropriately selected for the type of imaging work or the imaging location, and the usability is improved. Also in this case, the method of deriving the pitch angle may be changed based on the imaging type or the imaging location included in the order information, instead of based on the roll angle. Thus, the same usability as described above can be achieved. In other words, the method of calculating the angle information of the radiographic imaging device is changed (selected or determined), based on information of imaging to be performed (the type of imaging work or the imaging location). Also in this case, the method of calculating the angle information of the imaging device 1 may be changed, based on (i) the information of imaging to be performed (the type of imaging work and the imaging location) and (ii) the threshold value of the roll angle.

There is a case where the distance information of the distance measurer 29 is inaccurate owing to an insufficient directly exposed region (through region), so that the pitch angle cannot be derived or the accuracy thereof is decreased, based on the distance information. To deal with such a case, the method of deriving the pitch angle may be determined, based on information on the through region. Specifically, in a case where the selected imaging region, the information of which is included in the imaging order, does not allow a sufficient through region, the pitch angle calculated based on the gravitational acceleration information of the imaging device 1 is always used. On the other hand, in a case where the selected imaging region allows a sufficient through region, the pitch angle calculated based on the distance information of the distance measurer 29 is always used, or the method of deriving the pitch angle is changed between two methods depending on the roll angle, as described above. In other words, the method of calculating the angle information of the radiographic imaging device is changed (selected or determined), based on the information to be performed (imaging region). The imaging region information may be derived from optical image data captured by the optical imaging unit (optical camera) 2A that shows the region of the subject (subject S), instead of using the information on the imaging region included in the imaging order.

Instead of the imaging information, optical image data captured by the optical imaging unit (optical camera) 2A may be used to detect the through region; and based on the detection result, it may be determined whether a sufficient through region can be secured. When it is determined that a sufficient through region cannot be secured, the pitch angle calculated based on the gravitational acceleration information of the imaging device 1 is always used. On the other hand, when it is determined that a sufficient through region can be secured, the pitch angle calculated based on the distance information of the distance measurer 29 is always used, or the method of deriving the pitch angle is switched between two methods depending on the roll angle, as described above. Whether a sufficient through region can be secured may be determined, based on depth data of the depth camera instead the optical image data. The through region is captured by the depth camera as the irradiation surface of the imaging device 1. Therefore, the distribution of the depth data at the through region is the same as the distribution when a flat object is present. On the other hand, when the subject is captured by the depth camera, the distribution of the depth data of the subject is different from the distribution of a flat object. Thus, it is possible to identify the through region from the depth data. Further, the depth data and the optical image data may be combined. By the combination of the depth data and the optical image data, the through region is identified more accurately. In other words, the method of calculating the angle information of the radiographic imaging device is changed (selected or determined), based on the optical image data and/or the depth data of the depth camera.

Instead of changing the method of calculating the pitch angle between the two calculation methods depending on the roll angle (roll angle after switching of the imaging device 1), the angle information of the imaging device 1 calculated in step S6 and the angle information of the imaging device 1 calculated in step S9 may be weighted, based on the roll angle; and based on the weighted angle information, the angle information of the imaging device 1 with respect to the tube 23 may be calculated, or example.

In the above description, the method of deriving the pitch angle is changed, based on the roll angle of the imaging device 1. Instead, the roll angle of the generating device 2 (tube 23) may be used. Since the imaging device 1 can be freely carried, its roll angle changes to various angles particularly when the imaging device 1 is carried. Owing to the changes in the roll angle, the method of deriving the pitch angle may be frequently switched, which is not usable for the user. On the other hand, the roll angle of the tube 23 is stable and normally zero degree when not in use. Further, the user can intuitively grasp the relation between the movement of the tube 23 and the roll angle of the tube 23 when the user changes the position of the tube 23. By using the roll angle of the generating device 2 (the tube 23) to determine whether to change the derivation method, it is possible to reduce the frequency of changing the derivation method. Accordingly, the usability is improved. In other words, the method of calculating the angle information of the radiographic imaging device is changed (selected or determined), based on the gravitational acceleration information of the generating device 2.

Both the roll angle of the imaging device 1 and the roll angle of the generating device 2 (tube 23) may be used to determine whether to change the derivation method. For example, the method of deriving the pitch angle may be changed as described above when the roll angle of the imaging device 1 and the roll angle of the generating device 2 are both equal to or greater than a threshold value (e.g., 45 degrees). By using the roll angles of both the imaging device 1 and the generating device 2, the method of deriving the pitch angle is changed when the imaging device 1 and the generating device 2 are both close to their ideal positions. Accordingly, the frequency of changing the derivation method is reduced, and the usability is increased. Different threshold values may be used for the respective roll angles. In other words, the angle information of the radiographic imaging device is calculated, based on the distance information of the measurer (distance measurer 29), the gravitational acceleration information of the radiographic imaging device (imaging device 1), and/or the gravitational acceleration information of the generating device 2. In other words, the method of calculating the angle information of the radiographic imaging device is changed (selected or determined), based on the gravitational acceleration of the generating device 2 and the gravitational acceleration information of the radiographic imaging device.

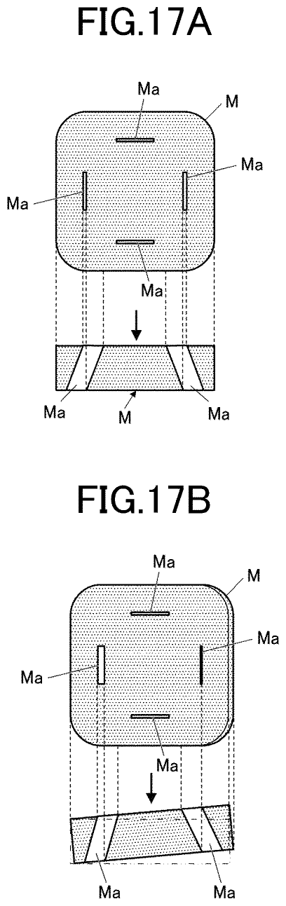

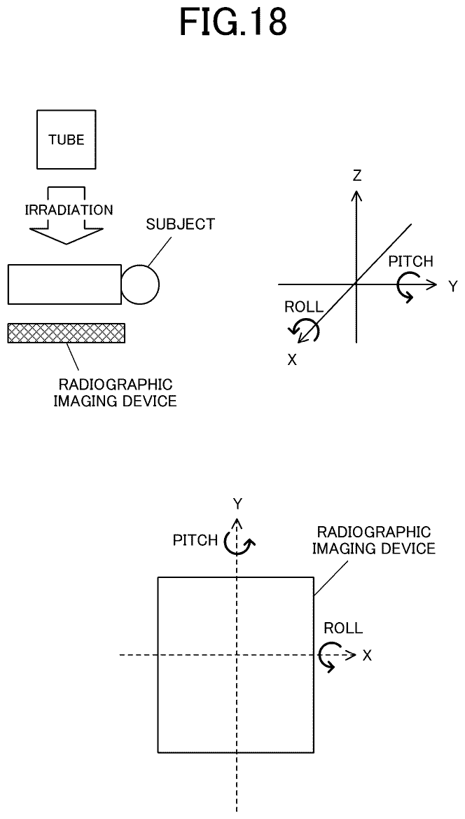

Further, as the above-described state determination process, the second controller 211 may detect the inclination of the subject S, output information on the detected inclination of the subject S, and determine whether the positioning of the subject S is appropriate, based on the information on the detected inclination of the subject S and the angle information of the imaging device 1. The result of the determination may be displayed on the sub display 28 or may be output as a warning message (sound) from the sound output section 33. The inclination of the subject S can be obtained by attaching a marker M to the subject S and capturing an image of the marker M attached to the subject S. FIG. 17A and FIG. 17B illustrate an example of the marker M that has through holes Ma at four positions on the upper, lower, left, and right sides. The through holes Ma penetrate toward the attachment surface when viewed in the irradiation direction of the radiation R. Each hole Ma is inclined to be farther from the center of the marker M as it extends toward the attachment surface. Although FIG. 17A and FIG. 17B illustrate only the cross-sectional view of the marker M in the right-left direction, the cross-sectional view of the marker M in the top-bottom direction is the same as in the right-left direction.

When the radiation R hits the marker M, the radiation R is attenuated while passing through part of the marker M other than the holes Ma. Accordingly, the radiation R that reaches the subject S or the imaging device 1 has a decreased dose. On the other hand, the radiation R that passes through the holes Ma is not attenuated. Accordingly, the radiation R that reaches the subject S or the imaging device 1 through the holes Ma has a higher dose than the radiation having passed through the part other than the holes Ma. Generally, in the radiographic image, a portion where the radiation R is greatly attenuated is whitish, whereas a portion where the radiation R is less attenuated is blackish. Therefore, part of the marker M other than the holes Ma is displayed in white, whereas part of the marker M corresponding to the holes Ma is displayed in black. When the marker M is disposed such that the attachment surface is perpendicular to the irradiation axis (optical axis) of the radiation R, the holes Ma formed at the left and right sides of the marker M incline in opposite directions but have the same inclination angle with respect to the irradiation axis of the radiation R. Therefore, the holes Ma (slits) on the right and left sides of the marker M have the equal width when viewed from the focal point side of the radiation R, as illustrated in FIG. 17A. On the other hand, when the marker M is disposed such that the attachment surface inclines to the irradiation axis of the radiation, the holes Ma on the right and left sides of the marker M have different inclination angles with respect to the irradiation axis. Accordingly, the holes Ma (slits) on the right and left sides of the marker M appear to have different widths when viewed from the focal point side of the radiation R, as illustrated in FIG. 17B. Specifically, the hole Ma located in a direction in which the marker M inclines appears large. The width of the hole Ma changes in proportion to the angle at which the attachment surface of the marker M inclines with respect to the irradiation axis. By using this principle, it is possible to calculate in which direction and by how much the marker M inclines. Thus, based on the inclination direction of the marker M, the inclination direction of the subject S wearing the marker M can be estimated, and the direction of the subject S can be adjusted in an appropriate direction for imaging.

In the state determination process described above, it is determined whether the angle information on the imaging device 1 relative to the tube 23 is within a predetermined reference range, based on the angle information calculated by the angle information calculation method selected from multiple angle information calculation methods in the angle information preparation process. According to such a method, the number of types of state determination results to be referred to by the user is reduced, so that the screen can be simplified, and the usability can be improved. On the other hand, the method of calculating the angle information of the imaging device 1 with respect to the tube 23 is changed (the definition and meaning of the angle information are changed) depending on the roll angle of the imaging device 1. Such a configuration may be difficult for a user who is not used to the system 100 to understand. To deal with this, instead of selecting one method from multiple angle information calculation methods in the angle information preparation process, multiple pieces of angle information may be calculated by the respective angle information calculation methods; all or part of the multiple pieces of angle information may be displayed; and the angle information as the target of state determination may be selected by using the method of selecting the angle information calculation method described above. Specifically, first, the second controller 211 calculates the angle information of the imaging device 1 with respect to the tube 23 by the first angle information calculation method (step S6 in FIG. 5) and the second angle information calculation method (step S9 in FIG. 5). The second controller 211 then displays, on the display (e.g., the sub display 28), all or part of the angle information of the imaging device 1 with respect to the tube 23 calculated by the first angle information calculation method and the second angle information calculation method. The second controller 211 determines whether the roll angle of the imaging device 1 (the roll angle after switching) is less than 45 degrees. When determining that the roll angle of the imaging device 1 is less than 45 degrees, the second controller 211 determines whether the angle information of the imaging device 1 relative to the tube 23 calculated by the first angle information calculation method is within a predetermined reference range. On the other hand, when determining that the roll angle of the imaging device 1 is 45 degrees or greater, the second controller 211 determines whether the angle information of the imaging device 1 relative to the tube 23 calculated by the second angle information calculation method is within a predetermined reference range. In the above, the angle information as the target of the determination is changed, based on the roll angle of the imaging device 1 in the above. The angle information as the target of the determination may be changed, based on the roll angle of the generating device 2, the imaging technique classification included in the order information, or the optical image data and/or the depth data. The angle information as the target of the determination may also be changed based on a combination of two or more of them.

In other words, the method of the determination on the angle information of the imaging device 1 with respect to the tube 23 is changed (angle information as the target of determination is changed), based on the information of the imaging to be performed. It can also be said that the method of the determination on the angle information of the imaging device 1 with respect to the tube 23 is changed (angle information as the target of determination is changed), based on the gravitational acceleration information of the imaging device 1 and/or the gravity acceleration information of the generating device 2 (tube 23). It can also be said that the method of the determination on the angle information of the imaging device 1 with respect to the tube 23 is changed (angle information as the target of determination is changed), based on the information on imaging to be performed, the gravitational acceleration information of the imaging device 1, and/or the gravity acceleration information of the generating device 2 (tube 23). It can also be said that the method of the determination on the angle information of the imaging device 1 with respect to the tube 23 is changed (angle information as the target of determination is changed), based on the optical image data and/or the depth data.

### [3-3-3. Display Control Process]

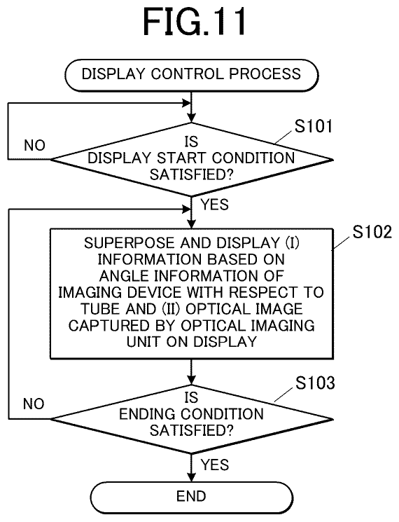

The second controller 211 performs a display control process as illustrated in FIG. 11 when performing the calculation process of step S6 or step S9 in the angle information preparation process, for example. The second controller 211 performs the display control process in parallel with the angle information preparation process.

When the display control process is started, the second controller 211 firstly determines whether at least one of the following display start conditions (1) to (5) is satisfied (step S101).
(1) An imaging order is selected at the console that instructs start of imaging.
(2) The imaging device 1 is taken out of a housing (e.g., the housing 26 if the system 100 is a medical cart; a charging cradle if the system 100 is installed in an imaging room).
(3) The imaging device 1 is removed from the cable.
(4) A predetermined button of the collimator 25 of the tube 23 is operated (e.g., a lamp button for emitting visible light to the irradiation field of the radiation R).
(5) The angle information of the imaging device 1 with respect to the tube 23 is within a reference range.

When determining in step S101 that neither of the display start conditions is met (step S101: NO), the second controller 211 repeats the determination process of step S101 (the second controller 211 waits until the display start condition is met). When determining that at least one of the display start conditions is met (step S101: YES), the second controller 211 proceeds to step S102.

When the user U is roughly adjusting the position and the direction of the tube 23, the imaging device 1 may not be placed under or behind the subject S yet, and the angle information may not be helpful. If the angle information is displayed at such timing, the user U may be confused. For example, the user U may adjust the direction of the tube 23 according to the angle of the imaging device 1 that has not been accurately positioned. By the determination process of step S101 by the second controller 211, at least either the main display 31 or the sub display 28 displays the angle information after at least one of the display start conditions is met. This can avoid giving confusion in the user U. Further, when the collimator 25 is emitting visible light, the user U is in the final stage of positioning, and the imaging device 1 is likely to be placed under or behind the subject S. By proceeding to the next step S102 when the display start condition (4) is met, the user U can receive useful angle information at appropriate timing.

In the determination process of step S101, the display start conditions may also include the following (6) to (8).
(6) After the imaging device 1 is taken out from the housing 26 (the inclination of the imaging device 1 starts changing), the inclination of the imaging device 1 becomes stable again (the imaging device 1 is arranged under or behind the subject S and is not moved).
(7) The distance (measurement value) measured by the distance measurer 29 becomes stable (the imaging device 1 is arranged under or behind the subject S and is not moved).
(8) A detection value of an air pressure sensor that is included in the imaging device 1 and that detects the air pressure in the casing of the imaging device 1 has exceeded a reference value (the imaging device 1 is placed under or behind the subject S, and the casing is pressed).
(9) The imaging device 1 is shown in an optical image generated by the optical imaging unit 2A.

The second controller 211 proceeds to the next step S102 when the display start condition (6), (7), or (8) is met, so that the second controller 211 displays the angle information on the display 28 and/or 31 after the imaging device 1 is placed under or behind the subject S. This can avoid giving confusion in the user U. Further, the fact that the image sensing device 1 is shown in the area imaged by the optical imaging unit 2A means that the imaging device 1 will be placed under or behind the subject S immediately thereafter. Therefore, by proceeding to the next step when the display start condition (9) is met, the second controller 211 displays the angle information on the display 28 and/or 31 at substantially the same timing as in the case of proceeding to the next step when the display start condition (6), (7), or (8) is met. This can avoid giving confusion to the user U.

Further, the same display start conditions and the same display end conditions may be applied for the multiple pieces of angle information: (i) angle information of the tube 23; (ii) angle information of the imaging device 1; (iii) the difference between (i) and (ii); and (iv) the determination result. For another example, the display start conditions and the display end conditions may be partly or entirely differentiated for the multiple pieces of angle information (i) to (iv). For example, when setting the position of the tube 23 before arranging the imaging device 1 behind the subject S, the angle information of the tube 23 may be sufficient. Therefore, to increase usability, display of the angle information (i) may be started at the start of the angle information preparation process, whereas display of (ii) to (iv) may be started when any of the display start conditions (1) to (9) is met. For another example, display of (i) may be started at the start of the angle information preparation process; display of (ii) and (iii) may be started when the display start condition (1) or (9) is met, and display of (iv) may be started when the display start condition (4) is met.

Returning to FIG. 11, after determining in step S101 that the display start condition is met, the second controller 211 performs the display process (step S102). In this display process, the second controller 211 displays an angle information display screen on at least either the main display 31 or the sub display 28. The angle information display screen shows (i) information based on the angle information of the imaging device 1 with respect to the tube 23 calculated in step S6 or step S9 of the angle information preparation process and (ii) the optical image captured by the optical imaging unit 2A such that (i) and (ii) are superposed. Herein, the information based on the angle information of the imaging device 1 with respect to the tube 23 includes the angle information itself of the imaging device 1 with respect to the tube 23 and an imaging device imitation image that imitates the imaging device 1. As described above, the angle information of the imaging device 1 with respect to the tube 23 is information on the angle of the imaging device 1 in the right-left direction and the up-down direction (i.e., in the roll direction and the pitch direction) with respect to the irradiation direction of the radiation emitted by the tube 23 (radiation emitter). The imaging device imitation image is an image that imitates the imaging device 1 as viewed such that the direction of sight coincides with the irradiation direction of the radiation emitted from the tube 23. When the imaging device imitation image is displayed, information on the device size (information regarding the size of the imaging device 1) included in the imaging conditions of the radiographic imaging may be acquired, and the display size of the imaging device imitation image may be determined, based on the information on the device size, for example. Thus, the size of the actually used imaging device 1 can be reflected on the imaging device imitation image. Thus, the user can easily have an image of the imaging device 1 from the displayed imaging device imitation image.

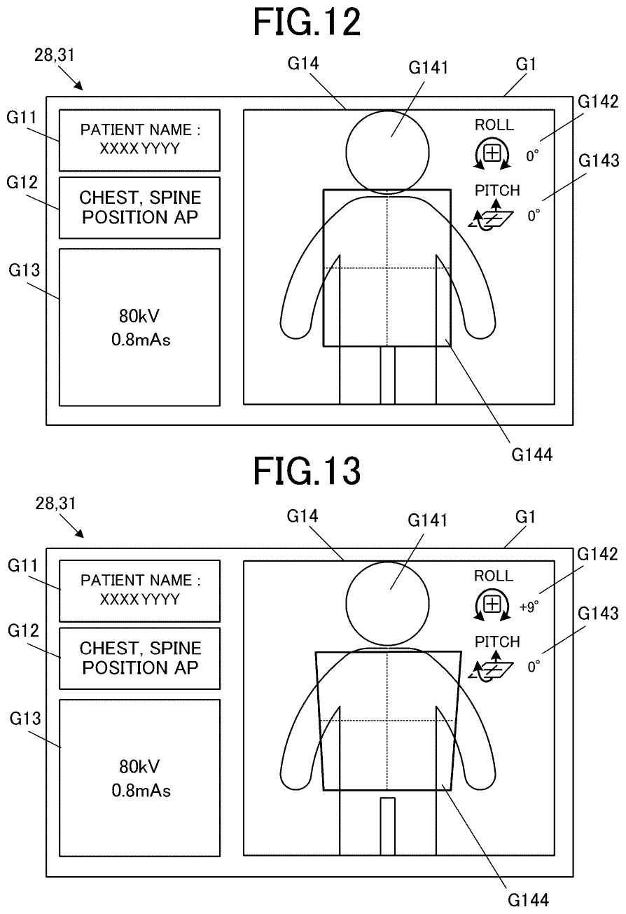

FIG. 12 to FIG. 15 are diagrams illustrating examples of the angle information display screen described above. As shown in FIG. 12 to FIG. 15, the angle information display screen G1 has four display sections: a first display section G11, a second display section G12, a third display section G13, and a fourth display section G14. The first display section G11 indicates the name of the subject S (patient name). The second display section G12 indicates the imaging region and the imaging direction that are imaging conditions of radiographic imaging. The third display section G13 indicates the tube voltage and the tube current-time product that are imaging conditions of radiographic imaging. The fourth display section G14 shows an optical image G141 that shows the subject S and that is obtained by the optical imaging unit 2A. The fourth display section G14 also shows angle information G142 and G143 on the right-left direction and the up-down direction (i.e., the roll and the pitch) of the imaging device 1 with respect to the irradiation direction of the radiation emitted by the tube 23 such that the angle information G142 and G143 are superposed on the optical image G141. The fourth display section G14 also shows the imaging device imitation image G144 such that the imaging device imitation image G144 is superposed on the optical image G141.

In the example of FIG. 12, both the angle information G142 in the roll direction and the angle information G143 in the pitch direction are “0°”. That is, the roll angle (angle information) of the imaging device 1 with respect to the horizontal plane is equal to the roll angle (angle information) of the tube 23 with respect to the horizontal plane. That is, the difference between the roll angle of the imaging device 1 and the roll angle of the tube 23 is 0°. Further, the pitch angle (angle information) of the image sensing device 1 with respect to the horizontal plane is equal to the pitch angle (angle information) of the tube 23 with respect to the horizontal plane. That is, the difference between the pitch angle of the imaging device 1 and the pitch angle of the tube 23 is 0°. That is, in the example of FIG. 12, the imaging device 1 and the tube 23 are arranged such that the irradiation axis of the radiations R emitted by the tube 23 is orthogonal to the radiation incident surface 1a of the imaging device 1. Therefore, in the example of FIG. 12, the imaging device imitation image G14 is shown in a rectangular shape in the fourth display section G144. That is, in the example of FIG. 12, the imaging device imitation image G144 imitates the imaging device 1 when the radiation incident surface 1a is viewed from the front.

In the example of FIG. 13, the angle information G142 on the roll direction is “+90” and the angle information G143 on the pitch direction is “0”. That is, the roll angle of the imaging device 1 is different from the roll angle of the tube 23 by +9°. Further, the difference between the pitch angle of the imaging device 1 and the pitch angle of the tube 23 is 0°. Therefore, in the example of FIG. 13, the imaging device imitation image G14 shown in the fourth display section G144 is rotated such that its upper part comes forward and its lower part goes backward in the screen. That is, the imaging device imitation image G144 in the example of FIG. 13 has a trapezoidal shape having the upper side longer than the lower side.

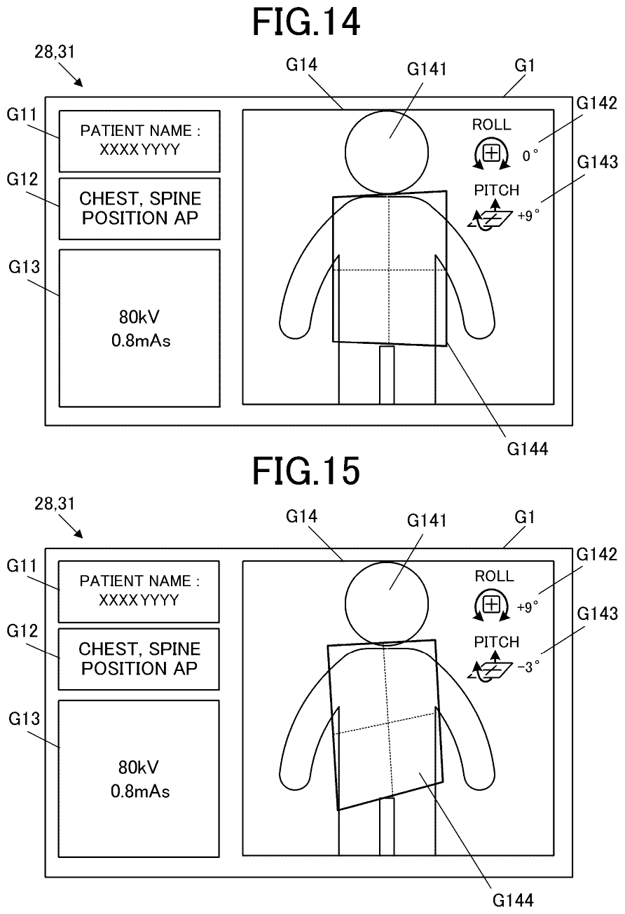

In the example of FIG. 14, the angle information G142 on the roll direction is “0”, and the angle information G143 on the pitch direction is “+9°”. That is, the difference between the roll angle of the imaging device 1 and roll angle of the tube 23 is 0°. The difference between the pitch angle of the imaging device 1 and the pitch angle of the tube 23 is +9°. Therefore, in the example of FIG. 14, the imaging device imitation image G14 shown in the fourth display section G144 is rotated such that its right part comes forward and its left part goes backward in the screen. That is, the imaging device imitation image G144 in the example of FIG. 14 has a trapezoidal shape having the right side longer than the left side.

In the example of FIG. 15, the angle information G142 on the roll direction is “+9°”, and the angle information G143 on the pitch direction is “−3°”. That is, the roll angle of the imaging device 1 is different from the roll angle of the tube 23 by +9°. Further, the difference between the pitch angle of the imaging device 1 and the pitch angle the tube 23 is −3°. That is, in the example of FIG. 15, the imaging device 1 and the tube 23 are arranged such that the irradiation axis of the radiations R emitted by the tube 23 inclines to the radiation incident surface 1a of the imaging device 1. Therefore, in the example of FIG. 15, the imaging device imitation image G14 in the fourth display section G144 is rotated such that its upper part comes forward, its lower part goes backward, its left part comes forward, and its right part goes backward in the screen. That is, the imaging device imitation image G144 in the example of FIG. 15 has a rectangular shape having the upper side longer than the lower side and having the left side longer than the right side.

In the fourth display section G14, the angle information G142 and G143 on the roll and pitch directions may be displayed in different colors depending on the numerical values indicated by the angle information G142 and G143. Specifically, when the numerical values indicated by the angle information G142 and G143 are within a reference range (e.g., ±30), the angle information G142 and G143 are displayed in black. On the other hand, when the numerical values indicated by the angle information G142 G143 are beyond the reference range (e.g., 3⁰), the angle information G142 and G143 are displayed in red. Similarly, in the fourth display section G14, the imaging device imitation image G144 may be displayed in a different color depending on the numerical values indicated by the angle information G142 and G143. Specifically, when the numerical values indicated by the angle information G142 and G143 are within a reference range (e.g., 3⁰), the imaging device imitation image G144 is displayed in black. On the other hand, when the numerical values indicated by the angle information G142 and G143 are beyond the reference range (e.g., ±30), the imaging device imitation image G144 is displayed in red.

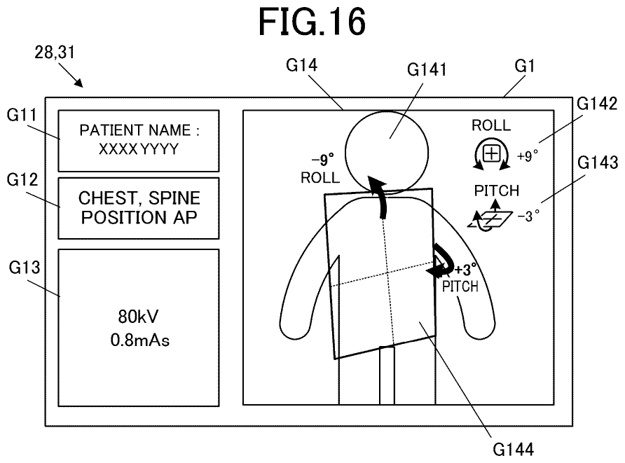

In the fourth display section G14, when the angle information G142 and G143 on the roll and the pitch directions are displayed, information for supporting the change of the angle information of the imaging device 1 with respect to the tube 23 may be displayed. Specifically, as shown in FIG. 16, when the numerical value indicated by the angle information G142 is +9°, that is, when the numerical value indicated by the angle information G142 is not 0°, information G145 is displayed for supporting the change of the angle information G142 such that the numerical value indicated by the angle information G142 becomes 0°. The example of FIG. 16 shows information G145 that suggests rotating the imaging device 1 by the roll angle of 9° in the minus direction so that the numerical value indicated by the angle information G142 becomes 0°. Similarly, when the numerical value indicated by the angle information G143 is −3°, that is, when the numerical value indicated by the angle information G143 is not 0°, information G146 is displayed for supporting the change of the angle information G143 such that the numerical value indicated by the angle information G143 becomes 0°. The example of FIG. 16 shows information G146 that suggests rotating the imaging device 1 by the pitch angle of 3° in the plus direction so that the numerical value indicated by the angle information G143 becomes 0°. When the information for supporting the change of the angle information of the imaging device 1 with respect to the tube 23 is displayed, the angle information may not be displayed. Further, the information for supporting the change of the angle information of the imaging device 1 with respect to the tube 23 may be displayed when the numerical value indicated by the angle information is out of the reference range. That is, when the numerical value indicated by the angle information is within the reference range, the information for supporting changing the angle information on the imaging device 1 relative to the tube 23 may not be displayed.

Further, when the imaging device imitation image G144 is displayed in the fourth display section G14, an imitation image (not shown) in an ideal state may also be displayed together. The imitation image in the ideal state represents how the imaging device 1 appears on the screen of the visible light camera when the imaging device 1 is positioned in an ideal state that is derived from the imaging conditions included in the imaging order. To display the imitation image in the ideal state, how the outline of the imaging device 1 appears on the visible light camera when the imaging device 1 is at the intended SID position and when the irradiation surface of the imaging device 1 is perpendicular to the X-ray axis is derived, by using the information on the intended SID and the panel size to be used in imaging, which are included in the imaging conditions. The outline of the imaging device 1 in the ideal state may be derived by calculations, based on the geometrical arrangement of the camera; may be derived by referring to a table of values obtained by experiments beforehand; or may be derived by combining the above calculations and table. The imitation image in the ideal state is displayed such that the center of the imitation image is at the center of the camera image, so that the processing can be simplified and implemented by low-speed processing hardware. The position of the X-ray axis at the target SID position may be calculated, and the center of the imitation image may be aligned with that position. For another example, the position of the X-ray axis at the target SID position may be stored in a table, and the center of the imitation image may be aligned with the position. The X-ray axis may not be aligned with the center of the imaging device 1, depending on the imaging region. Therefore, information regarding the display position of the imitation image in the ideal state may be stored for each imaging order or each imaging region, and the display position of the imitation image may be determined, based on the information. Since oblique imaging is not frequently performed, the shape of the imitation image in the ideal state is basically a square or a rectangle. When the panel of the imaging device 1 to be used in imaging has the aspect ratio of 1 (e.g., 17 inches×17 inches), the imitation image in the ideal state has a square shape. When the aspect ratio is not 1 (e.g., 17 inches×14 inches), it has a rectangular shape. In the latter case, whether the currently used imaging device 1 is oriented horizontally or vertically may be recognized from the optical image captured by the optical imaging unit 2A; and the orientation of the imitation image in the ideal state may be aligned with the recognized orientation. Thus, the usability is increased. Whether the imaging device 1 is oriented horizontally or vertically may be recognized, based on the gravitational acceleration information of the imaging device 1 or the combination of the image and the gravitational acceleration information. Since it is difficult to determine the orientation of the imaging device 1 from the gravitational acceleration information in spine position imaging, it is effective to combine the image and the gravitational acceleration information. The imitation image in the ideal state may correspond to oblique incidence. The oblique incidence angle is acquired from the imaging direction and the imaging technique type included the imaging order information; and the outer circumference of the imaging device 1 when the imaging device 1 is placed at the oblique incidence angle at the target SID is derived by calculations. For another example, the outer circumference of the imaging device 1 is derived by referring to a table or by combining the above calculations and the table.

In the fourth display section G14, if the framing of the imaging device 1 appearing in the optical image G141 is shifted from the framing of the imaging device imitation image G144, usability is decreased. It is desirable to adjust the display of the imaging device imitation image G144 so that the framing of the imaging device imitation image G144 matches with the outer shape of the imaging device 1 appearing in the optical image G141. To adjust the display of the imaging device imitation image G144, the position and the size of the displayed imaging device imitation image G144 are adjusted, for example. More specifically, it is desirable that the following measure A or measure B is taken. Measure A: the size and the position of the imaging device 1 on the image are recognized using one or more sides among the four sides of the imaging device 1 or using two or more corners among the four corners of the imaging device 1 in the optical image G141; and the recognized set of sides or corners (e.g., two pairs of corners) is matched with the corresponding sides or corners of the framing of the imaging device imitation image G144, so that the size and the position of the framing of the displayed imaging device imitation image G144 are matched with the size and the position of the imaging device 1 appearing in the optical image G141. It is desirable to adjust both the display size and the display position of the framing of the imaging device imitation image G144. However, the sides or corners of the imaging device 1 may not stably appear in the optical image G141 depending on the user or the patient during positioning, and the display size or the display position may not be adjusted. In such a case, the imaging device imitation image G144 is displayed at a predetermined display position in a predetermined display size (measure B). As the predetermined display size, the size of the panel that appears in the camera when the imaging device 1 is at a standard SID (e.g., 100 cm or 120 cm) in imaging with a medical cart may be derived by calculations, for example. The predetermined display position may be a position at which the center of the camera image is aligned with the center of the imaging device imitation image G144. As the display position of the imaging device imitation image G144, the position of the imaging region of the patient appearing in the camera image may be recognized, based on information on the imaging region to be imaged; and based on the recognized position, the position of the imaging device imitation image G144 may be determined. Either the measure A or the measure B may be selected, based on the result of image recognition of the optical image G141 displayed in the fourth display section G14.

Returning to FIG. 11, after performing the display process in step S102, the second controller 211 performs the end determination process (step S103). In the end determination process, the second controller 211 determines whether at least one of the following end conditions (1) to (3) is satisfied.
(1) The irradiation instruction switch 22 has been operated.
(2) The irradiation with the radiation R has been completed.
(3) The angle information preparation process has ended.

In the end determination process, when determining that neither of the end conditions is not satisfied (step S103: NO), the second controller 211 returns to step S102 and repeats the processing therefrom. That is, the second controller 211 continues the display process of step S102 until the end condition is satisfied. On the other hand, when determining that the end condition has been met (step S103: YES), the second controller 211 ends the display control process.

### [3-3-4. Display Control Process and Others]

In the display control process described above, the second controller 211 may execute a confirmation process before starting the determination process in step S101. In this confirmation process, the second controller 211 determines whether the imaging device 1 has received an instruction to start imaging from the console 3. If there are multiple imaging devices 1 registered in the console 3 (housed in the housing 26), the second controller 211 executes this confirmation process for each imaging device 1. Further, in this case, in the subsequent display process of step S102, the second controller 211 displays the angle information of the imaging device 1 that has received an imaging start instruction from the console 3 on the display 28 and/or 31. There are cases where multiple imaging devices 1 of different sizes are mounted on a medical cart. If angle information of the multiple imaging devices 1 is displayed simultaneously, the user U may be confused. By the confirmation process by the second controller 211, the user U is allowed to know which angle information is displayed among the multiple imaging devices 1.

In the display control process, when the second controller 211 displays the angle information (the angle information of the imaging device 1 with respect to the tube 23) on the display 28 and/or 31 during imaging preparations before imaging the subject S, the second controller 120 may display the angle information in the past (the angle information of the imaging device 1 with respect to the horizontal plane) when the subject S was imaged in the past on the display 28 and/or 31. Specifically, the second controller 211 calls the past angle information, based on the ID of the subject S, and causes the display 28 and/or 31 to display the past angle information. In this case, it is preferable that the second controller 211 calls the past angle information, based on the ID and imaging region (e.g., chest, abdomen) of the subject S. This is because the inclination of the imaging device 1 may differ depending on the imaging region. If the inclination of the tube 23 or the subject S changes every time of imaging, the arrangement of the internal structure of the subject S or the density of the radiographic image may change. Owing to such changes, the user may overlook a minute change at the time of follow-up observation. With the past angle information, the positioning is highly reproducible, and the risk of overlooking a minute change can be reduced. Further, when the angle information display screen is displayed, an imaging device imitation image in the past (not illustrated) may be generated from the past angle information and displayed together with the above-described imaging device imitation image G144. The user U can perform positioning such that the imaging device imitation image G144 matches with the past imaging device imitation image. Thus, the user U can easily perform imaging at the same angle as in the past. The imaging device imitation image G144 and the past imaging device imitation image may have different colors or different types of lines from each other. Further, the past imaging device imitation image may be generated as an imitation image in an ideal state. To generate the past imaging device imitation image as an imitation image in an ideal state, the angle information in the past imaging is used as the angle information in the oblique incidence of the imitation image in the ideal state.

Further, the second controller 211 may superpose the past angle information and/or the SID (SSD) on the radiographic image. Thus, the limited display space may be effectively used, and the user U does not have to move his/her eyes frequently between the radiographic image and the past angle information in preparing for imaging or making diagnosis. When the currently prepared imaging is to be performed in the supine position or the standing position, the second controller 211 may not display the past angle information on the display 28 and/or 31. In spine position imaging or standing position imaging, it is obvious that the angle formed by the horizontal plane and the radiation incidence plane 1a is 0° or 90°. Therefore, displaying the past angle information may rather confuse the user U.

### [3-3-5. Generation of Radiation]

When the second controller 211 receives an operation signal from the irradiation instruction switch 22 (when the irradiation instruction switch 22 is operated), the second controller 102 transmits, to the generating device 213, an irradiation instruction signal that instructs the generator 213 to generate the radiation R in a mode corresponding to a radiographic image to be generated (a still image or a dynamic image consisting of multiple frames). When receiving the irradiation instruction signal from the second controller 211, the generator 213 applies a voltage corresponding to the imaging conditions set beforehand to the tube 23 and supplies a current corresponding to the imaging conditions to the tube 23. When receiving the voltage and the current from the generator 213, the tube 23 generates the radiation R having a dose corresponding to the applied voltage and the supplied current in a mode corresponding to the applied voltage and the supplied current. In capturing a still image, the tube 23 emits the radiation R one time per press of the irradiation instruction switch 22. In capturing a dynamic image, the tube 23 repeats emitting the pulsed radiation R multiple times per predetermined time (e.g., 15 times per second) or continues emitting the radiation R for a predetermined time per press of the emission instruction switch 22.

### [3-3-6. Storing Angle Information]

After generating radiation (performing imaging), the second controller 211 performs a storing process. In the storing process, the second controller 211 stores the angle information at the time of imaging the subject S (the angle information of the imaging device 1 and the tube 23 with respect to the horizontal plane) together with the information on the subject S. The angle information may be stored by writing the angle information in a header portion of the radiographic image or by storing the angle information in the second storage section 212 or a storage section of a different device (e.g., PACS) in association with the radiographic image, for example. In this storing process, the second controller 211 may store not only the angle information but also the SID (SSD) at the time of imaging the subject S together. By storing the angle information together with the SID, the user U can check the SID in the past imaging of the subject S when performing a new session of imaging, and the user can reproduce the positions and orientations of the imaging device 1 and the tube 23 accurately in the new session of imaging. In particular, if the angle information and the SID are written in the header portion of the radiographic image (the radiographic image and the angle information are associated with each other), the angle information can be more efficiently managed. Further, the angle information can be utilized more usefully in diagnosis. For example, a doctor can easily imagine the arrangement of the internal body structure that should be present.

When multiple radiographic images are generated per one time of imaging operation (e.g., dynamic imaging, serial imaging), in the storing process, the second controller 211 may store the angle information (the angle information of the imaging device 1 and the tube 23 with respect to the horizontal plane) corresponding to the respective radiographic images (frames) at the time of obtaining the respective radiographic images. In this way, the user U or a person who makes diagnosis can check whether there is a large body motion at the time of imaging by comparing multiple pieces of angle information. Further, by referring to the multiple pieces of angle information, the user U can easily and automatically delete an abnormal radiographic image from multiple radiographic images or remove the abnormal radiographic image from analysis targets. Further, displaying the angle information together with the radiographic image can attract the attention of the person who makes diagnosis. Further, a graph for the multiple radiographic images (frames) may be displayed having the horizontal axis representing the time axis and the vertical axis representing the angle information. Thus, a frame corresponding to the angle change can be easily found without checking through the frames. Herein, the graph may be displayed in parallel with a play bar (seek bar). By sliding the cursor of the play bar and stopping the cursor at a desired position, a frame image corresponding to the position can be displayed. In such a case, it is preferable that the horizontal axis of the graph is aligned with the play bar. According to such a configuration, the user U can slide the cursor to the position (time point) where the angle is changed to display the frame image corresponding to the angle change. Such a configuration can be intuitively used and convenient. Further, an angle criterion may be prepared, and a point of the graph corresponding to a frame exceeding the angle criterion may be visually differentiated. Such a graph is easy to understand. For another example, the graph may not be displayed, and a point (time point) of the play bar corresponding to the frame where the angle exceeds the angle criterion may be visually differentiated. Such a configuration eliminates the need for a dedicated display section, simplifies the appearance of the display, and improves usability. Further, the angle criterion may be a threshold value of the angle with respect to the tube 23 or may be a change amount, a ratio, a slope of change, or a shape (pattern) of change between the angles in two or more frames. When dynamic imaging (serial imaging) is performed, pieces of angle information (angle information of the imaging device 1 and the tube 23 with respect to the horizontal plane) corresponding to the respective radiographic images (frames) at the time of capturing the respective radiographic images are judged. When the angle information changes by a predetermined value or greater, the change may be displayed on the display 28 and/or 31. The judgement of the angle information may be performed by comparing the angle information with angle information in the past capturing of images (past imaging). When the angle information corresponding to the respective radiographic images at the time of capturing the respective radiographic images is different from the angle information in the past imaging by a predetermined value or greater, the difference may be displayed on the display 28 and/or 31.

When pieces of angle information (angle information of the imaging device 1 and the tube 23 with respect to the horizontal plane) corresponding to the respective radiographic images at the time of capturing the respective radiographic images are stored, the second controller 211 may execute the determination process in the storing process. In the determination process, the second controller 211 determines representative angle information that represents the imaging, based on the pieces of angle information stored for the respective radiation images. The representative angle information representing the imaging may include: angle information at the time of generating a radiographic image at a predetermined number among the multiple radiographic images; an average value of all pieces of angle information; a median value of all pieces of angle information; and an average value of part of all pieces of angle information (the angle information corresponding to the generation of the first radiographic image and the last radiographic image), for example. Handling multiple pieces of angle information is troublesome for the user U and a person who makes diagnosis. According to the above configuration, the user U or the diagnosing person can refer to only the representative angle information in positioning or diagnosis, so that the user U or the diagnosing person can reduce their work. Further, if an average value or the like is used as the representative angle information, the representative angle information can reflect the imaging situations accurately. Further, if an average value, a median value, or the like is used as the representative angle information, it is possible to exclude the influence of variations in the angle information caused by breathing or the like when the representative angle information is referred to in positioning or diagnosis.

### [3-3-7. Operation of Radiation Generating Device and Others]

According to the above embodiment, the second controller 211 causes the display 28 and/or 31 to display the angle information when the display start condition is satisfied while repeatedly performing the calculation process of step S6 or step S9 in the angle information preparation process (see FIG. 5). However, the calculation process of step S6 or step S9 in the angle information preparation process may be started when the display start condition is satisfied. According to this, it is possible to suppress the power consumption of the second controller 211, as compared to the case where the calculation process of step S6 or step S9 in the angle information preparation process is executed before the display start condition is satisfied. In this case, after starting the calculation process of step S6 or step S9 in the angle information preparation process, the second controller 211 may not display the angle information on the display 28 and/or 31 but may only store the angle information. According to this, the user U can check whether there is any abnormality in imaging by referring to the angle information at the time of maintenance, for example.

When there are multiple medical carts of the same type, the inclination angle of the housing 26 of the medical cart may slightly differ from each other due to individual differences of the medical carts (e.g., distortion of the casing of the medical cart). To deal with this, the second controller 211 may correct (calibrate) values detected by the first sensor 17 received from the imaging device 1 housed in the housing 26. Specifically, the second controller 211 of each medical cart corrects the output values so that the output values indicate that the rotation angle (inclination angle) of the imaging device 1 with respect to the horizontal plane is identical among all the medical carts when the imaging device 1 is housed in the housing 26.

### <4. Effect>

As described above, the system 100 according to the present embodiment includes the tube 23 (radiation emitter) that emits the radiation R. The system 100 includes the distance measurer 29 (measurer) that measures the distance between the tube 23 and the imaging device 1 and outputs distance information. The imaging device 1 generates an image, based on the radiation R emitted by the tube 23. Based on the distance information measured by the distance measurer 29 (measurer), the system 100 calculates angle information of the imaging device 1 with respect to the irradiation direction of the radiation R emitted by the tube 23.

According to the system 100, the angle information of the imaging device 1 with respect to the irradiation direction of the radiation R emitted by the tube 23 is calculated, based on the distance information measured by the distance measurer 29. Therefore, it is possible to appropriately calculate the angle information of the imaging device 1 regardless of the posture of the disposed imaging device 1. Therefore, even when the radiation R is horizontally emitted from the tube 23, the angles of the tube 23 and the imaging device 1 can be appropriately adjusted.

### <5. Others>

The above-described embodiment is not intended to limit the present invention and canbe modified without departing from the spirit and scope of the present invention. The scope of the present invention should be interpreted by terms of the appended claims.

For example, although a hard disk, a semiconductor nonvolatile memory, or the like is used in the above description as a computer-readable medium storing the program according to the present invention, the present invention is not limited to this example. Other applicable computer-readable media include portable recording media such as CD-ROM. In addition, a carrier wave is also applied as a medium for providing data of the program according to the present invention via a communication line.

1. A radiographic imaging system comprising:
an optical camera that obtains an optical image;
a display that displays the optical image obtained by the optical camera;
a radiation emitter that emits radiation to a radiographic imaging device that generates a radiographic image; and
a hardware processor, wherein:
the hardware processor calculates angle information of the radiographic imaging device in a right-left direction and an up-down direction with respect to a direction of the radiation emitted by the radiation emitter, and
the hardware processor superposes predetermined information on the optical image and displays the predetermined information and the optical image on the display, the predetermined information being based on the calculated angle information of the radiographic imaging device in the right-left direction and the up-down direction.

2. The radiographic imaging system according to claim 1, wherein:
based on the calculated angle information, the hardware processor compares an angle of the radiographic imaging device in the right-left direction and an angle of the radiographic imaging device in the up-down direction, and
based on the comparison, the hardware processor switches between the angle information in the right-left direction and the angle information in the up-down direction.

3. The radiographic imaging system according to claim 1, wherein
based on the angle information, the hardware processor changes a color of the angle information that is included in the predetermined information and that is displayed on the display.

4. The radiographic imaging system according to claim 1, wherein:
the predetermined information includes an imitation image that imitates the radiographic imaging device, and
based on the angle information, the hardware processor changes a color of the imitation image displayed on the display.

5. The radiographic imaging system according to claim 1, wherein:
the predetermined information includes an imitation image that imitates the radiographic imaging device, and
based on the angle information, the hardware processor changes a shape of an outer framing of the imitation image displayed on the display.

6. The radiographic imaging system according to claim 1, wherein
based on the angle information, the hardware processor displays information that supports adjusting an angle of the radiographic imaging device in the right-left direction or the up-down direction on the display.

7. The radiographic imaging system according to claim 1, wherein
when an angle of the radiographic imaging device in the right-left direction or the up-down direction indicated by the angle information is changed by a predetermined value or greater during radiographic imaging by the radiographic imaging device, the hardware processor notifies that the angle is changed.

8. The radiographic imaging system according to claim 7, wherein
as the radiographic imaging, the radiographic imaging device performs serial imaging in which a frame constituting a moving image is repeatedly generated.

9. The radiographic imaging system according to claim 1, wherein the radiographic imaging system is a movable medical cart.

10. The radiographic imaging system according to claim 1, wherein:
the radiographic imaging device includes a gravitational acceleration sensor capable of measuring gravitational acceleration and outputs gravitational acceleration information,
the system further includes a measurer that measures a distance between the radiation emitter and the radiographic imaging device and outputs distance information, and
based on the distance information of the measurer and/or the gravitational acceleration information of the radiographic imaging device, the hardware processor calculates the angle information of the radiographic imaging device in the right-left direction and the up-down direction.
